(19) United States
(12) Patent Application Publication
(10) Pub. No.: US 2008/0208324 A1
(43) Pub. Date: Aug. 28, 2008

(54) METHOD AND APPARATUS FOR OCCLUDING AN ANATOMICAL STRUCTURE

(76) Inventors: Jason I. Glithero, Mason, OH (US); Livyn O. Okorocha, Cincinnati, OH (US); James G. Tappel, Cincinnati, OH (US); Eric E. Bielefeld, New Albany, IN (US); Michael W. Goldenbogen, Floyd Knobs, IN (US); Chadd R. Tyler, Sellersburg, IN (US); Todd A. Hall, Goshen, KY (US); Stephen C. Robinson, Milford, OH (US); Greg R. Furnish, Louisville, KY (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 12/033,935

(22) Filed: Feb. 20, 2008

Related U.S. Application Data
(60) Provisional application No. 60/891,298, filed on Feb. 23, 2007.

(51) Int. Cl. A61F 2/06; A61B 17/08

(52) U.S. Cl. 623/1.36; 606/151; 623/1.3

(57) ABSTRACT

Methods and apparatus for occluding an anatomical structure including a device having at least first and second clamp portions adapted to cooperate to move from an open position adjacent an anatomical structure to a closed position engaging and occluding the anatomical structure. A system for delivery of a fluid to affect occlusion of an anatomical structure is provided, as well as an applicator for deploying occlusion devices having different sizes.

### CROSS REFERENCE TO RELATED APPLICATIONS

This application claims the benefit of U.S. Provisional Application Ser. No. 60/891,298, filed Feb. 23, 2007, the contents of which are incorporated herein by reference.

### BACKGROUND

Embodiments of the present invention relate to devices and methods that may be used to occlude a hollow anatomical structure. Examples of occlusion devices and associated methods are described in U.S. application Ser. No. 10/853,928, filed on May 26, 2004, U.S. Application Ser. No. 60/699,309 filed on Jul. 14, 2005; and International Application Serial No. PCT/US2006/027553, published as WO 2007/009099, and filed on Jul. 14, 2006, each of which are incorporated by reference herein. For instance, it may be desirable to occlude an anatomical structure such as, for example, the left atrial appendage (LAA), in a manner sufficient to prevent the entry of blood therein. It will be appreciated that blood may otherwise tend to clot in a non-occluded LAA, which may increase the risk of stroke. Occlusion of the LAA may prevent blood from entering the LAA, thereby preventing blood from clotting in the LAA. Such occlusion may therefore prevent blood clots from exiting the LAA into the blood stream, such that the risk of stroke may be reduced by occluding the LAA. Of course, there may be other purposes for occluding the LAA, and there are a variety of other anatomical structures that may be occluded. Due to the varying dimensions of the LAA and other anatomical structures between individuals, it will be appreciated that it may be desirable to provide occlusion devices of varying dimensions and/or configurations to correspond to the particular anatomical structure intended to be occluded or for other purposes. While a variety of techniques and devices have been used to treat anatomical structures such as the LAA, it is believed that no one prior to the inventors has previously made or used an invention as described below.

### BRIEF DESCRIPTION OF DRAWINGS

It is believed the present invention will be better understood from the following description of certain examples taken in conjunction with the accompanying drawings, in which like reference numerals identify the same elements and in which:

FIG. 1A depicts a cross-sectional view of an anatomical structure, specifically a left atrial appendage and left atrium;

FIG. 1B depicts a cross-sectional view of an exemplary use of an occlusion device on the anatomical structure of FIG. 1A;

FIG. 2 depicts a front, cross-sectional view of an exemplary occlusion device;

FIG. 3A depicts a plan view of an alternative occlusion device; FIG. 38 depicts a plan view of the occlusion device of FIG. 3A including end caps;

FIG. 3C depicts a front view of the occlusion device of FIG. 38 in a closed configuration;

FIG. 3D depicts a front view of the occlusion device of FIG. 38 in an open configuration and an exemplary applier;

FIG. 4A depicts a perspective view of an alternative occlusion device in an open configuration;

FIG. 4B depicts a front view of the occlusion device of FIG. 4A in an open configuration with a barb plate in a placement configuration;

FIG. 4C depicts a front view of the occlusion device of FIG. 4A in an open configuration with a barb plate in a deployed configuration;

FIG. 4D depicts a front view of the occlusion device of FIG. 4A in a closed configuration with a barb plate in a placement configuration;

FIG. 4E depicts a front view of the occlusion device of FIG. 4A in a closed configuration with a barb plate in a deployed configuration;

FIG. 5 depicts a perspective view of an alternative occlusion device;

FIG. 6 depicts a perspective view of an alternative occlusion device in an open configuration;

FIG. 7A depicts a perspective view of a sample of material usable in an alternative method of occluding an anatomical structure;

FIG. 7B depicts a cross-sectional view of the material of FIG. 7A being drawn into an insertion device;

FIG. 7C depicts a cross-sectional view of the insertion device of FIG. 7B filled with the material of FIG. 7A;

FIG. 7D depicts a perspective view of the insertion device of FIG. 7B piercing an anatomical structure;

FIG. 7E depicts a cross-sectional view of the insertion device of FIG. 7B depositing the material of FIG. 7A into the anatomical structure of FIG. 7D;

FIG. 8A depicts a perspective view of an alternative occlusion device; FIG. 8B depicts a cross-sectional view of the occlusion device of FIG. 8A;

FIG. 9 depicts a perspective view of an alternative occlusion device in an open configuration;

FIG. 10 depicts a partial cross-sectional view of the occlusion device of FIG. 9 in an open configuration;

FIG. 11 depicts a perspective view of an alternative occlusion device in an open configuration;

FIG. 12 depicts a cross-sectional view of the occlusion device of FIG. 11 in a closed configuration engaging an anatomical structure;

FIG. 13 depicts a perspective view of an alternative occlusion device in an open configuration;

FIG. 14 depicts a cross-sectional view of the occlusion device of FIG. 13 in a closed configuration engaging an anatomical structure;

FIG. 15 depicts a perspective view of an alternative occlusion device in a closed configuration;

FIG. 16 depicts a front view of the occlusion device of FIG. 15 in an open configuration;

FIG. 17A depicts a perspective view of an alternative occlusion device in a closed configuration;

FIG. 17B depicts a front view of the occlusion device of FIG. 17A in an open configuration;

FIG. 18A depicts a partial cross-sectional view of the hinge of the occlusion device of FIG. 17A;

FIG. 18B depicts a partial cross-sectional view of an alternative hinge of the occlusion device of FIG. 17A;

FIG. 18C depicts a partial cross-sectional view of an alternative hinge of the occlusion device of FIG. 17A;

FIG. 19 depicts a perspective view of an alternative occlusion device in an open configuration;

FIG. 20 depicts a front view of an alternative occlusion device in a partially open configuration;

FIG. 21 depicts a partial perspective view of the locking portion of the occlusion device of FIG. 20;

FIG. 22 depicts a front view of an alternative occlusion device in an open configuration;

FIG. 23 depicts a partial perspective view of the locking portion of the occlusion device of FIG. 22 in a closed configuration;

FIG. 24 depicts a front view of an alternative occlusion device; FIG. 25 depicts a bottom view of the occlusion device of FIG. 24;

FIG. 26 depicts a perspective view of an alternative occlusion device in an open configuration;

FIG. 27A depicts a front view of the occlusion device of FIG. 26 in an open configuration;

FIG. 27B depicts a front view of the occlusion device of FIG. 26 in a closed configuration;

FIG. 28 depicts a top view of an alternative occlusion device;

FIG. 29 depicts a front view of the occlusion device of FIG. 28 in a partially open configuration;

FIG. 30A depicts a perspective view of an alternative occlusion device in a closed configuration;

FIG. 30B depicts a cross-sectional view of the occlusion device of FIG. 30A in a closed configuration;

FIG. 31 depicts a perspective view of an alternative occlusion device in a closed configuration;

FIG. 32A depicts an end view of the occlusion device of FIG. 31 in a closed configuration;

FIG. 32B depicts an end view of the occlusion device of FIG. 31 in an open configuration;

FIG. 33 depicts a perspective view of an alternative occlusion device in an open configuration;

FIG. 34 depicts a front view of the occlusion device of FIG. 33 engaging an anatomical structure as the device is transitioned from an open configuration toward a closed configuration with an exemplary applier;

FIG. 35 depicts a perspective view of an alternative method of occluding an anatomical structure and exemplary components associated with that method;

FIG. 36 depicts a perspective view of an alternative method of occluding an anatomical structure and exemplary components associated with that method;

FIG. 37 depicts a perspective view of an anatomical structure occluded according to the method of FIG. 36;

FIG. 38 depicts a perspective view of an alternative occlusion device in an open configuration;

FIG. 39 depicts a partial cross-sectional view of the occlusion device of FIG. 38;

FIG. 40 depicts a perspective view of an alternative-occlusion device in an open configuration;

FIG. 41 depicts a front view of the occlusion device of FIG. 40 in a deflated closed configuration;

FIG. 42 depicts a front view of the occlusion device of FIG. 40 in an inflated closed configuration;

FIG. 43 depicts a partial perspective view of an exemplary applier; and

FIG. 44 depicts an alternative occlusion device with loops configured to engage an applier.

### DETAILED DESCRIPTION

The following description of certain examples of the invention should not be used to limit the scope of the present invention. Other examples, features, aspects, embodiments, and advantages of the invention will become apparent to those skilled in the art from the following description, which includes by way of illustration, one of the best modes contemplated for carrying out the invention. As will be realized, the invention is capable of other different aspects, all without departing from the invention. Accordingly, the drawings and descriptions should be regarded as illustrative in nature and not restrictive.

It will be appreciated that many of the following embodiments of an occlusion device may act as a clamp or clip. For instance, as will be described in greater detail below, an occlusion device may be secured to an anatomical structure. To the extent that such an anatomical structure would otherwise permit communication of fluid through the anatomical structure, the clamping or other engagement of an occlusion device on the anatomical structure may substantially prevent the communication of fluid through, into, or out of the anatomical structure. Many of the following embodiments of an occlusion device may therefore be used to form an occlusion in the anatomical structure. It will be appreciated, however, that an occlusion device need not necessarily be used to form a complete occlusion in an anatomical structure, and may be instead used simply to restrict or regulate the flow of fluid through, into, or out of an anatomical structure. It will also be appreciated that all of the following embodiments of an occlusion device may have a variety of alternative uses other than forming occlusions or restrictions in anatomical structures, and their use need not even be limited to use with anatomical structures. In addition, it will be appreciated that all of the following embodiments of an occlusion device may be configured such that the occlusion device is atraumatic with regard to the anatomical structure being occluded, adjacent organs, and/or adjacent tissue. Due to the varying dimensions of the LAA and other anatomical structures between individuals, it will be appreciated the overall dimensions or configuration of many of the following embodiments of an occlusion device may be varied to accommodate anatomical structures of different dimensions or for other purposes.

Many of the following embodiments of an occlusion device may include a sock configured to enshroud at least some components of the occlusion device. A sock may comprise a knit, braided polyester material. Of course, any other suitable materials may be used for a sock, including but not limited to polyethylene. It will also be appreciated that a sock is optional. Where an occlusion device is positioned adjacent an anatomical structure, a sock may be configured to provide friction and to facilitate the growth of scar tissue. A sock may also be sutured to tissue to further secure an occlusion device in place. The manner in which a sock may be incorporated in many of the following embodiments of occlusion devices and associated methods will become apparent to those of ordinary skill in the art. Alternatively, components of an occlusion device may be provided with a textured surface, or some other device or configuration may be used to provide results similar to those provided by a sock.

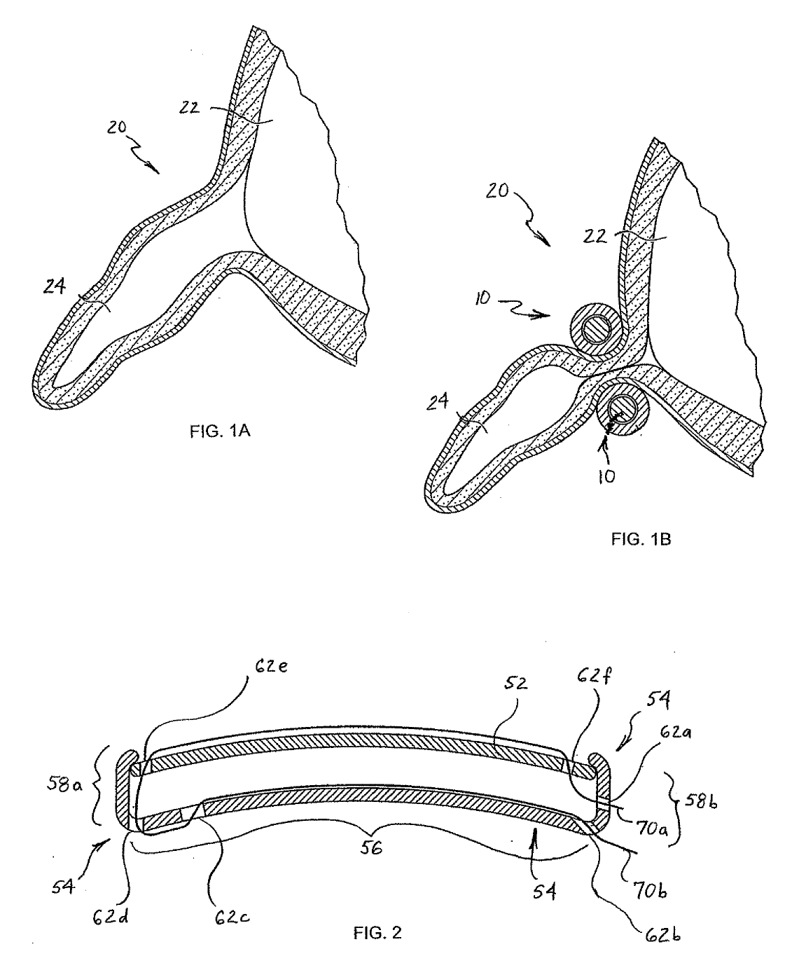

FIGS. 1A and 1B illustrate one exemplary use for an occlusion device (10). In particular, FIG. 1A shows a view of the left atrium (22) of a patient's heart (20). A left atrial appendage (LAA) (24) is shown extending from the left atrium (22). With an occlusion device (10) in an open configuration, the LAA (24) may be passed through the opening in occlusion device (10), and the occlusion device (10) positioned adjacent to the outside of the LAA (24). With occlusion device (10) so positioned, an applier (not shown) may release occlusion device (10), resulting in occlusion device (10) transitioning toward a closed configuration. FIG. 1B shows occlusion device (10) in a closed configuration. With occlusion device (10) being positioned as shown in FIG. 1B in a closed configuration, occlusion device (10) may be configured to exert between approximately 2.5 pounds per square inch (psi) and approximately 3 psi of pressure on LAA (24). Due to the configuration of occlusion device (10) in this particular example, such exerted pressure may be substantially uniform along the length of occlusion device (10). Of course, occlusion device (10) may be configured to exert any other suitable amount of pressure. In addition, occlusion device (10) may be configured such that pressure exerted by occlusion device (10) is not substantially uniform.

In the present example, force or pressure exerted by the closed occlusion device (10) results in occlusion device (10) creating an occlusion of the LAA (24) by clamping the LAA (24), thereby preventing blood from entering or leaving the LAA (24) relative to the left atrium (22). Accordingly, those of ordinary skill in the art will appreciate that occlusion device (10) may be used in a remedial or prophylactic fashion, particularly for reducing the risk of stroke by preventing the formation of blood clots in the LAA (24).

If occlusion device (10) is left in the position and configuration shown in FIG. 1B for a substantial period of time, the LAA (24) may simply atrophy and wither away, and the occlusion device (10) may essentially become engulfed in scar tissue. It will be appreciated that the use of occlusion device (10) as illustrated in FIGS. 1A and 18, and as described above, is merely exemplary, and that an occlusion device (10) may be used in a variety of different ways and with a variety of different anatomical structures or other structures. It will also be appreciated that any of the other occlusion devices described herein, or variations thereof, may be used in a manner similar to that illustrated in FIGS. 1A and 1B, and as described above, or in a variety of other ways. Various other suitable uses will be apparent to those of ordinary skill in the art.

FIG. 2 illustrates an exemplary occlusion device (50). As shown, occlusion device (50) comprises a horizontal member (52) and a base member (54). Horizontal member (52) and base member (54) are generally curved in this example. It will be appreciated that such curvature may compliment curvature of an anatomical structure to which occlusion device (50) will be secured, or may otherwise facilitate or optimize engagement of occlusion device (50) with an anatomical structure. Alternatively, horizontal member (52) and/or base member (54) may be generally straight or have any other suitable configuration. In this example, base member (54) comprises a horizontal portion (56) and vertical portions (58a and 58b) formed in a substantially u-shaped configuration.

In the illustrated version, horizontal member (52) and base member (54) include apertures (62a, 62b, 62c, 62d, 62e and 62f) through which a cinching device (70) is threaded. In this example, occlusion device (50) includes six apertures (62a-62f); however occlusion device (50) may include any suitable number of apertures in any suitable configuration. Cinching device (70) may comprise a thread looped through apertures (62a-62f) such that a first end (70a) and a second end (70b) extend through apertures (62a and 62b), respectively. Cinching device (70) may comprise a thread, a string, a band, or any other suitable method or device.

With horizontal member (52) substantially vertically separated from horizontal portion (56) of base member (54), occlusion device (50) may be considered to be in an open configuration. Alternatively, occlusion device (50) may be regarded as being in a closed configuration when horizontal member (52) is adjacent to horizontal portion (56), or where horizontal member (52) and horizontal portion (56) are positioned such that the vertical separation is relatively small. Occlusion device (50) may be transitioned from an open configuration toward a closed configuration by applying a substantially horizontal force to first end (70a), substantially perpendicularly to vertical portion (58b), while holding second end (70b) substantially stationary. Alternatively, occlusion device (50) may be urged toward a closed configuration by holding first end (70a) substantially stationary while applying a substantially horizontal force to second end (70b) substantially perpendicularly to vertical portion (58b). The transition of occlusion device (50) from an open configuration to a closed configuration may result in a positive lock. As used throughout this specification, the term “positive lock” refers to a one-way process of closing and locking a device, such that once the device reaches a closed configuration, the device may not be transitioned back to an open configuration without damaging the device. Other variations in structure, use, and method of operation of occlusion device (50) will be apparent to those of ordinary skill in the art, and some embodiments permit unlocking.

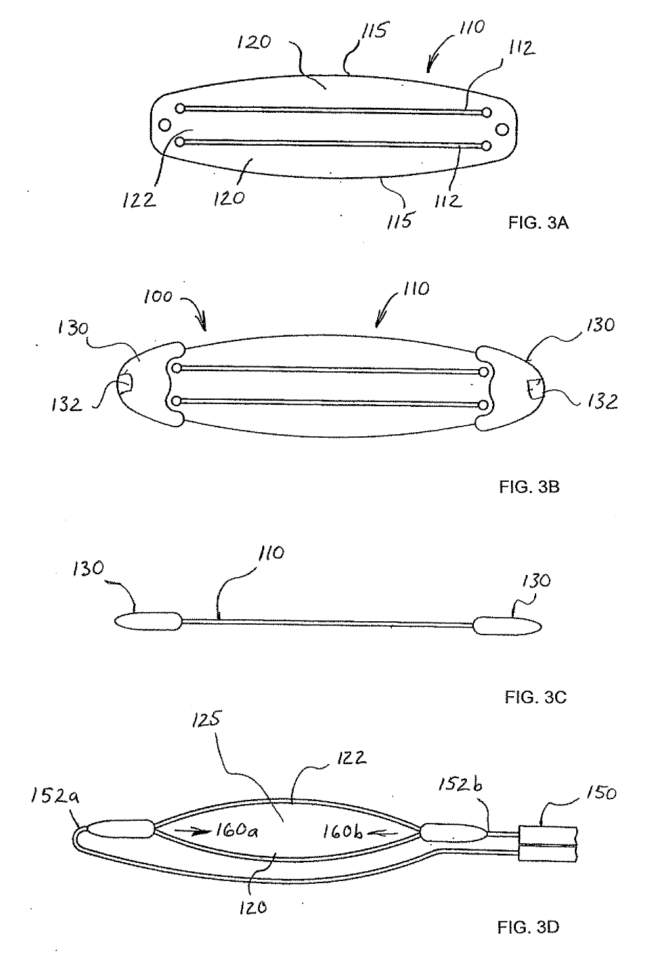

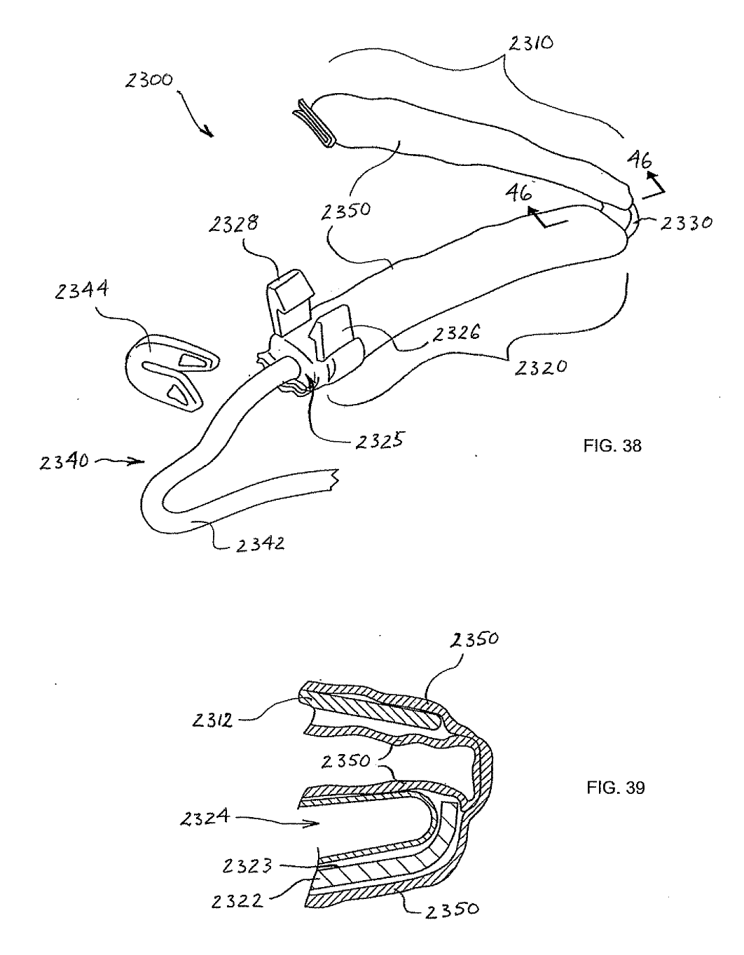

Another exemplary occlusion device (100) is shown in FIGS. 3A-3D. In this version, occlusion device (100) comprises a clamping member (110) and end caps (130). Clamping member (110) may comprise a substantially flat spring material having a generally rectangular cross-section. Clamping member may comprise Nitinol, plastic, or any other suitable material. As shown in FIGS. 3A-3D, clamping member (110) includes slots (112), a middle portion (122) positioned between slots (112), and outer portions (120) positioned between the respective slots (112) and the outer edges (115). In this example, clamping member (110) includes two slots (112) extending longitudinally along clamping member (110), however, any suitable number or configuration of slots (112) may be used. As shown in FIG. 38 end caps (130) are configured to be positioned on opposite ends of clamping member (110) and include notches (132). End caps (130) may comprise plastic, rubber, or any other suitable material, and end caps (130) may be formed using a molding processes or any other suitable device or method. As shown in FIG. 3C, when middle portion (122) and outer portions (120) form a substantially flat, co-planar surface, occlusion device (100) may be regarded as being in a fully closed configuration. Alternatively, as shown in FIG. 3D, when middle portion (122) is deflected in one direction and outer portions (120) are deflected in an opposite direction, thereby creating an annular opening (125), occlusion device (100) may be regarded as being in an open configuration.

As shown in FIG. 3D, occlusion device (100) is transitioned toward an open position utilizing an applier (150). Applier (150) may include urging members (152a, 152b) configured to be received by notches (132) in end caps (130). Once urging members (152a, 152b) are received by notches (132), urging members (152a, 152b) may exert opposing, substantially horizontal forces inward, in the directions of arrows (160a, 160b), respectively. As shown in FIG. 3D, the force may be sufficient to deflect outer portions (120) in a first direction, while deflecting middle portion (122) in the opposite direction, thereby transitioning occlusion device (100) to an open configuration. When occlusion device (100) is in an open configuration, an anatomical structure may be passed through the annular opening (125). Once the anatomical structure and occlusion device (100) are properly positioned, the substantially horizontal force may be removed, allowing occlusion device (100) to return toward the fully closed configuration, thereby occluding the anatomical structure. Other variations in structure, use, and method of operation of occlusion device (100) will be apparent to those of ordinary skill in the art.

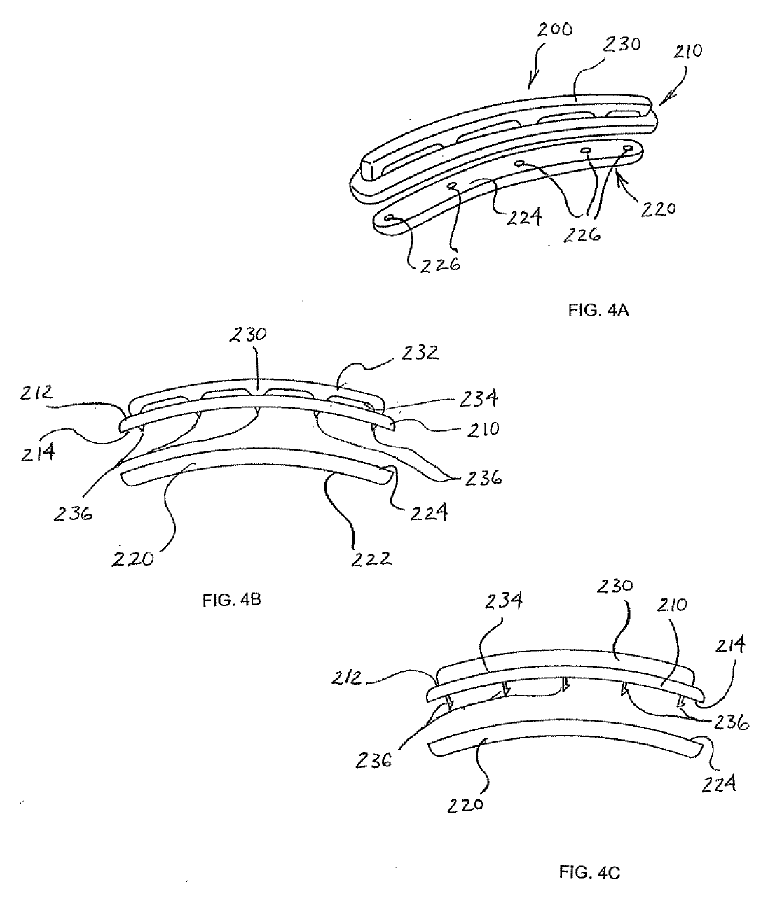

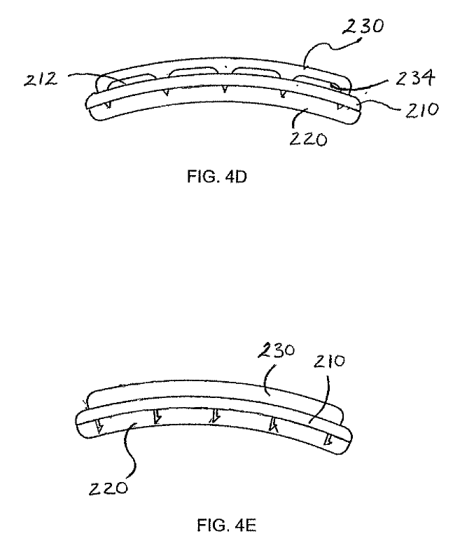

An alternative embodiment of an occlusion device (200) is shown in FIGS. 4A-4E. In this version, occlusion device (200) comprises an upper pressure plate (210); a lower pressure plate (220), and a barb plate (230). Occlusion device (200) may comprise plastic or any other suitable material, including, but not limited to, bioabsorbable polymers. As shown in FIGS. 4A-4E, upper pressure plate (210) includes an outer surface (212) and an inner surface (214). Barb plate (230) may include an outer surface (232), an inner surface (234), and barbs (236). Barbs (236) may project substantially vertically downward from inner surface (234). In the illustrated version, occlusion device (200) includes five barbs (236) equally spaced longitudinally along barb plate (230). However, occlusion device (200) may include any suitable number of barbs (236) in any suitable configuration. Upper pressure plate (210) may be configured such that barbs (236) may project through apertures (not shown) in upper pressure plate (210). As shown in FIGS. 4A-4E, lower pressure plate (220) includes an outer surface (222) and an inner surface (224). Inner surface (224) may include a plurality of receiving apertures (226). The number and configuration of receiving apertures (226) may correspond to the number and configuration of barbs (236). Lower pressure plate (220) may include any suitable alternative number of receiving apertures (226) in any suitable configuration.

Upper pressure plate (210), lower pressure plate (220), and barb plate (230) are generally curved in this example. It will be appreciated that such curvature may compliment curvature of an anatomical structure to which occlusion device (200) will be secured, or may otherwise facilitate or optimize engagement of occlusion device (200) with an anatomical structure. Alternatively, upper pressure plate (210), lower pressure plate (220) and barb plate (230) may be generally straight or have any other suitable configuration. In this example, upper pressure plate (210), lower pressure plate (220) and barb plate (230) have corresponding configurations, but that is not required. For example, upper pressure plate (210) and lower pressure plate (220) may be curved and barb plate (230) may be generally straight, or any other suitable combination of configurations may be used.

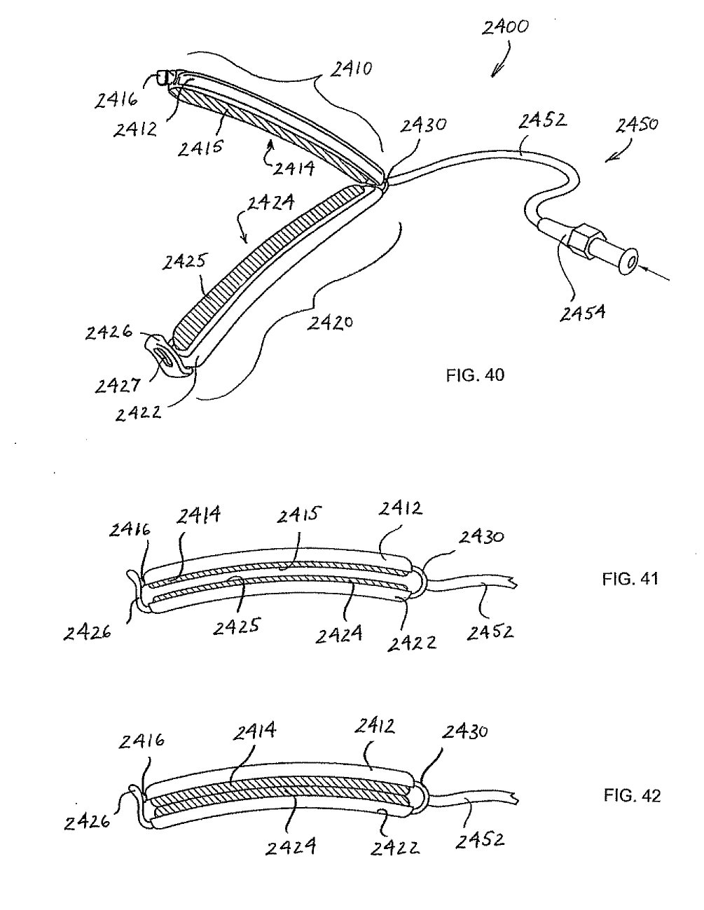

As shown in FIG. 4B, with inner surface (234) of barb plate (230) separated from outer surface (212) of upper pressure plate (210), barb plate (230) may be regarded as being in a placement configuration. As shown in FIG. 4C, when a substantially downward force is applied to barb plate (230), such that inner surface (234) of barb plate (230) is adjacent to outer surface (212) of upper pressure plate (210), barb plate (230) may be regarded as being in a deployed configuration. In an exemplary use of occlusion device (200), an anatomical structure (not shown) may be positioned between upper pressure plate (210) and lower pressure plate (220) while barb plate (230) is in a placement configuration. Once the anatomical structure is aligned, upper pressure plate (210) is positioned adjacent to lower pressure plate (220), as shown in FIG. 40. The anatomical structure may be occluded by applying a substantially downward force on barb plate (230), thereby transitioning barb plate (230) into a deployed configuration, as shown in FIG. 4E. Occlusion device (200) may be provided with a feature that permits upper pressure plate (210) to be removably attached to lower pressure plate (220) with barb plate (230) in a placement configuration or with barb plate (230) in a deployed configuration. For instance, a clasp, clip, or other device, mechanism, or feature may be provided on upper pressure plate (210) and/or lower pressure plate (220). In one embodiment, barbs (236) and receiving apertures (226) are configured such that engagement of barbs (236) within receiving apertures (226) provides a positive lock, thereby securing barb plate (230) and upper pressure plate (210) relative to lower pressure plate (220). It will be appreciated that other methods of use may be suitable, such as, for example, transitioning barb plate (230) into a deployed configuration prior to positioning upper pressure plate (210) adjacent to lower pressure plate (220). With upper pressure plate (210) adjacent to lower pressure plate (220), the transition of barb plate (230) from a placement configuration to a deployed configuration may result in a positive lock. Other variations in structure, use, and method of operation of occlusion device (200) will be apparent to those of ordinary skill in the art.

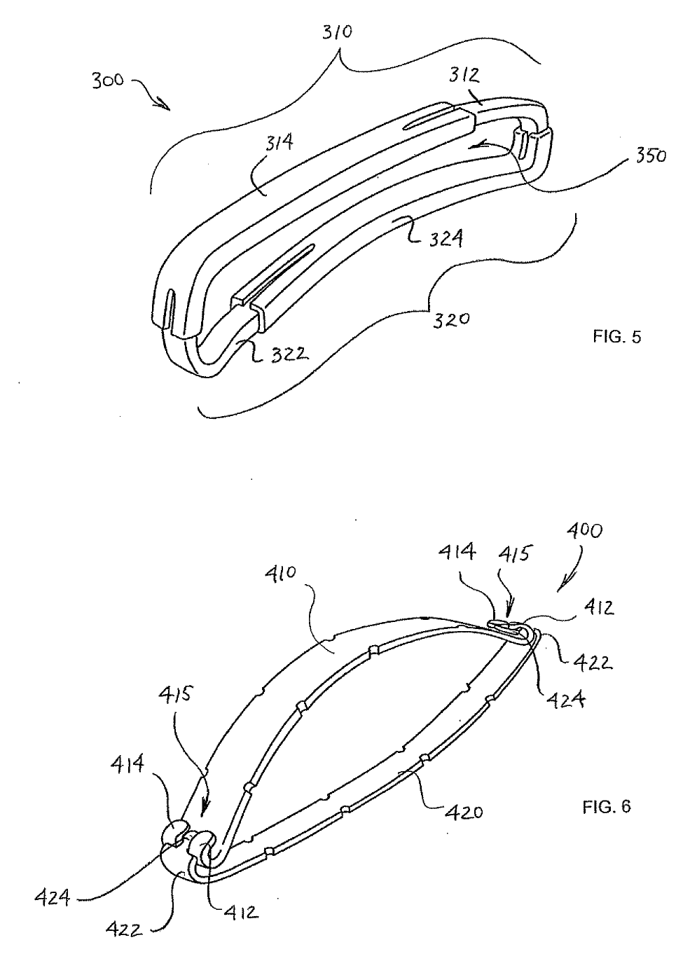

Another exemplary occlusion device (300) is shown in FIG, 5. In this version, occlusion device (300) comprises an upper portion (310) attached to a lower portion (320). Upper portion (310) may comprise an upper male member (312) inserted into an upper female member (314). Lower portion (320) may comprise a lower male member (322) inserted into a lower female member (324). As shown in FIG. 5, upper portion (310) is attached to lower portion (320) by inserting lower male member (322) into upper female member (314) and inserting upper male member (312) into lower female member (324). In the illustrated version, such attachment creates an opening (350) suitable for an anatomical structure. Male members (312 and 322) and female members (314 and 324) may be configured to operate as slide ratchets, thereby enabling the user to adjust the size of opening (350) horizontally and/or vertically to accommodate anatomical structures of varying lengths and/or thicknesses. By way of example only, such ratcheting may permit the size of opening (350) to only be reduced, without permitting it to be increased, such that the ratcheting provides a positive lock. Occlusion device (300) may comprise various metallic or nonmetallic materials or both. A suitable nonmetallic material would be plastic, such as a polycarbonate, but other suitable nonmetallic materials will be apparent to one of ordinary skill in the art. Male members (312 and 322) and female members (314 and 324) may comprise the same material or a combination of different materials.

Upper portion (310) and lower portion (320) are generally curved in this example. It will be appreciated that such curvature may compliment curvature of an anatomical structure to which occlusion device (300) will be secured, or may otherwise facilitate or optimize engagement of occlusion device (300) with an anatomical structure. Alternatively, upper portion (310) and lower portion (320) may be generally straight or have any other suitable configuration. In this example, upper portion (310) and lower portion (320) have corresponding configurations, but that is not required. For example, upper portion (310) and/or lower portion (320) may be generally straight or have any other suitable configuration. Other variations in structure, use, and method of operation of occlusion device (300) will be apparent to those of ordinary skill in the art.

An alternative embodiment of an occlusion device (400) is shown in FIG. 6. Occlusion device (400) comprises an upper clamping portion (410) and a lower clamping portion (420). Upper clamping portion (410) and lower clamping portion (420) may comprise a flat spring material, such as, for example, Nitinol, having a generally rectangular cross-section, however, other suitable materials and cross-sections may be used. Upper clamping portion (410) and lower clamping portion (420) may comprise the same material, but that is not required. In the illustrated version, upper clamping portion (410) includes a first hook (412) and a second hook (414) separated by a gap (415) at each distal end of upper clamping portion (410). As shown in FIG. 6, lower clamping portion (420) includes a rounded end (422) and tab (424) at each distal end of lower clamping portion (420). Upper clamping portion (410) may be attached to lower clamping portion (420) by inserting tab (424) into gap (415), such that tab (424) is positioned within first hook (412) and second hook (414). In order to complete the attachment, the attachment steps may be conducted on both distal ends of upper clamping portion (410) and lower clamping portion (420). Occlusion device (400) may be closed by forcing the center of upper clamping portion (410) (or some other portion(s) of upper clamping portion) toward lower clamping portion (420). Such movement of upper clamping portion (410) may result in upper clamping portion (410) “popping” or “snapping” toward lower clamping portion (420) after upper clamping portion (410) is moved a particular distance toward lower clamping portion (420). To the extent that a portion of an anatomical structure is placed between upper clamping portion (410) and lower clamping portion (420) at such time, the anatomical structure may be crimped between the “popped”/“snapped” upper clamping portion (410) and lower clamping portion (420). Other variations in structure, use, and method of operation of occlusion device (400) will be apparent to those of ordinary skill in the art.

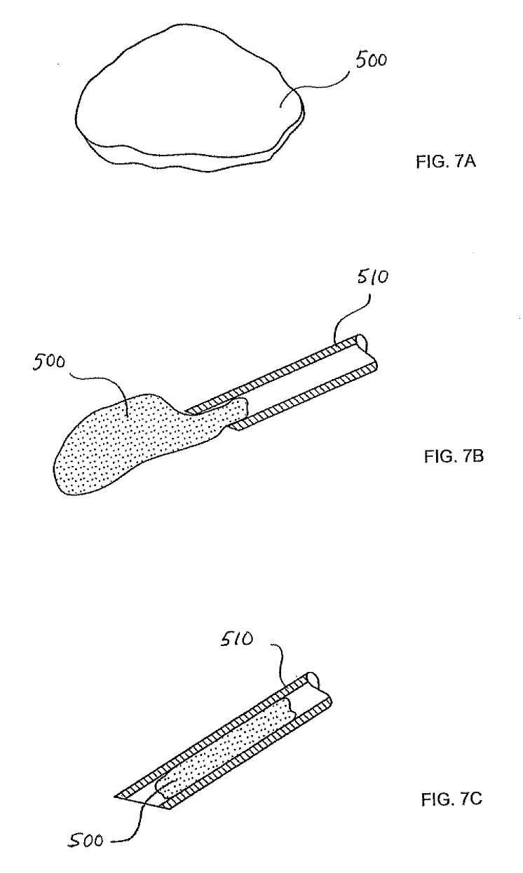

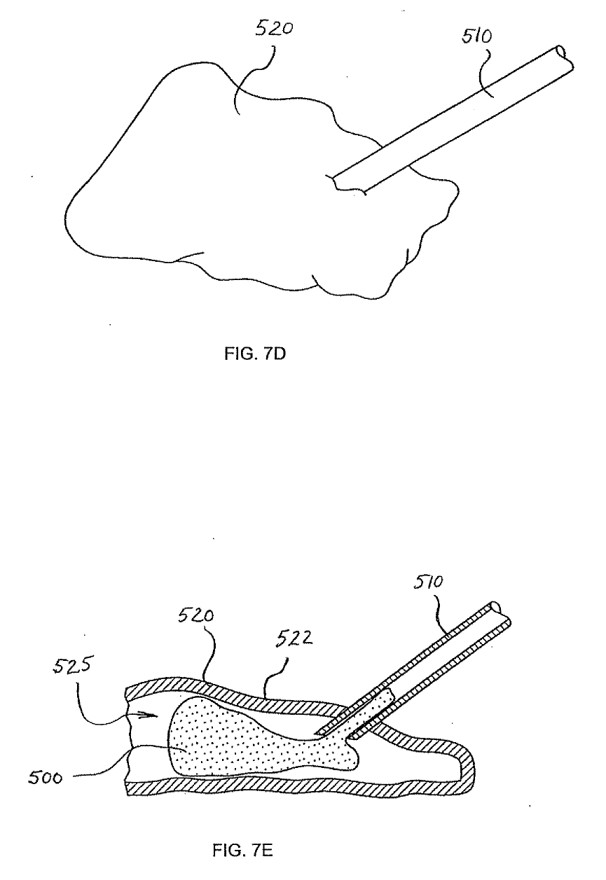

FIGS. 7A-7E depict a method and device for filling an anatomical structure (520) with a material (500). In the illustrated version, the method comprises drawing material (500) into an insertion device (510), piercing the outer wall (522) of anatomical structure (520) with the filled insertion device (510), and injecting material (500) from insertion device (510) into an interior cavity (525) of anatomical structure (520). Material (500) may at least partially occupy interior cavity (525), thereby at least partially occluding anatomical structure (520) (e.g., an LAA). Material (500) may comprise any suitable biocompatible material, including but not limited to an adhesive or collagen. Insertion device (510) may comprise a hypodermic needle, a suitable cannula, or any other suitable device. Other variations in structure, use, and method of operation of the components depicted in FIGS. 7A-7E will be apparent to those of ordinary skill in the art.

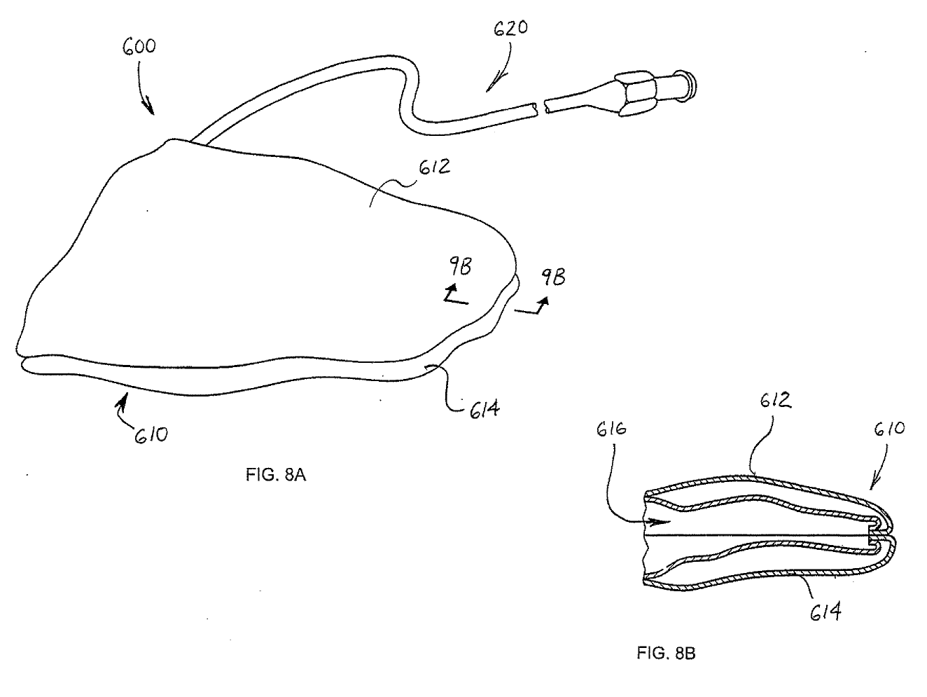

Another exemplary occlusion device (600) is shown in FIGS. 8A-8S. In the illustrated version occlusion device (600) comprises a bladder (610) connected to a fluid delivery system (620). Fluid delivery system (620) may include a luer fitting (not shown) or any other suitable device. As shown in FIGS. 8A-8S, bladder (610) includes an upper portion (612) and a lower portion (614) which may be separated to create a pocket (616) between upper portion (612) and lower portion (614). The shape and size of bladder (610) may be adapted to correspond to the anatomical structure being treated. An anatomical structure may be inserted into pocket (616) with upper portion (612) and (614) empty or partially filled. Fluid may be delivered through fluid delivery system (620) into upper portion (612) and/or lower portion (614) until the anatomical structure has been sufficiently occluded by upper portion (612) and lower portion (614). This embodiment may be particularly useful when attempting to treat anatomical structures having irregular shapes and/or thicknesses, or under other circumstances. Other variations in structure, use, and method of operation of occlusion device (600) will be apparent to those of ordinary skill in the art.

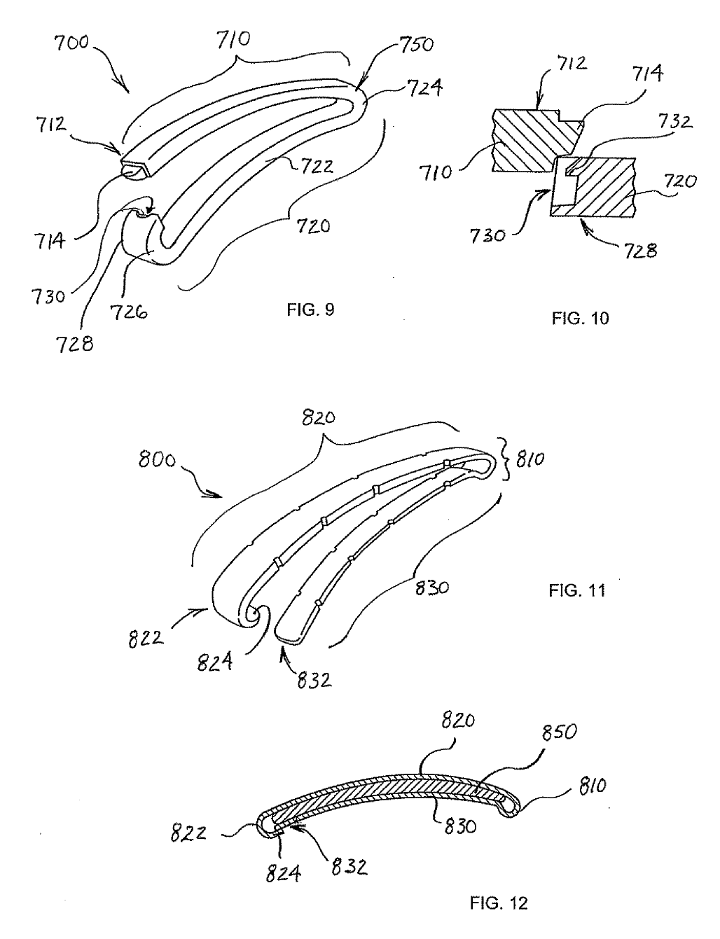

An alternative example of an occlusion device (700) is shown in FIGS. 9-10. In the illustrated version, occlusion device (700) has a unitary construction and comprises an upper clamping member (710) connected to a lower clamping member (720) with a living hinge (750). As used throughout this specification, the term “living hinge” refers to a thin section of material configured to connect two components and bend at the same time to allow the components to move. A living hinge may comprise plastic, metal, or any other suitable material. In this example, upper clamping member (710) includes a locking tab (714) positioned on free end (712) of upper clamping member (710). Lower clamping member (720) includes a horizontal portion (722) and two curved portions (724 and 726). Curved portion (724) may be positioned adjacent to living hinge (750). In the illustrated version, curved portion (726) is positioned at free end (728) of lower clamping member (720) and includes a recess (730). Recess (730) may be configured to receive locking tab (714). As shown in FIG. 10, a stop tab (732) is formed in recess (730), such that when locking tab (714) is received by recess (730), stop tab (732) engages locking tab (714). The engagement of locking tab (714) may result in a positive lock between upper clamping member (710) and lower clamping member (720). Occlusion device (700) may comprise metal, a polymer, such as, for example, polyglycolic acid, or any other suitable material.

Upper clamping member (710) and lower clamping member (720) are generally curved in this example. It will be appreciated that such curvature may compliment curvature of an anatomical structure to which occlusion device (700) will be secured, or may otherwise facilitate or optimize engagement of occlusion device (700) with an anatomical structure. Alternatively, upper clamping member (710) and lower clamping member (720) may be generally straight or have any other suitable configuration. In this example, upper clamping member (710) and lower clamping member (720) have corresponding configurations, but that is not required. For example, upper clamping member (710) and/or lower clamping member (720) may be generally straight or have any other suitable configuration. Other variations in structure, use, and method of operation of occlusion device (700) will be apparent to those of ordinary skill in the art.

Another exemplary occlusion device (800) is shown in FIGS. 11-12. In the illustrated version, occlusion device (800) has a unitary construction and comprises a hinge (810) connecting an upper clamping portion (820) and a lower clamping portion (830). Occlusion device (800) may comprise a metal, such as, for example, Nitinol, or any other suitable material. In this example, lower clamping portion (830) includes a free distal end (832), and upper clamping portion includes a curved distal end (822) with a lip (824). As shown in FIG. 11, occlusion device (800) is in an open configuration, while FIG. 12 depicts occlusion device (800) in a closed configuration. As shown in FIG. 12, when occlusion device (800) is in a closed configuration, free distal end (832) engages lip (824), thereby at least partially occluding an anatomical structure (850) positioned between upper clamping portion (820) and lower clamping portion (830).

Upper clamping portion (820) and lower clamping portion (830) are generally curved in this example. It will be appreciated that such curvature may compliment curvature of an anatomical structure to which occlusion device (800) will be secured, or may otherwise facilitate or optimize engagement of occlusion device (800) with an anatomical structure. Alternatively, upper clamping portion (820) and lower clamping portion (830) may be generally straight or have any other suitable configuration. In this example, upper clamping portion (820) and lower clamping portion (830) have corresponding configurations, but that is not required. For example, upper clamping portion (820) and/or lower clamping portion (830) may be generally straight or have any other suitable configuration. Other variations in structure, use, and method of operation of occlusion device (800) will be apparent to those of ordinary skill in the art.

An alternative embodiment of an occlusion device (900) is shown in FIGS. 1314. In the illustrated version, occlusion device (900) comprises an upper clamping member (910) attached to a lower clamping member (920) with a hinge (930). Upper clamping member (910) and lower clamping member (920) are generally curved in this example. It will be appreciated that such curvature may compliment curvature of an anatomical structure to which occlusion device (900) will be secured, or may otherwise facilitate or optimize engagement of occlusion device (900) with an anatomical structure. Alternatively, upper clamping member (910) and lower clamping member (920) may be generally straight or have any other suitable configuration. In this example, upper clamping member (910) and lower clamping member (920) have corresponding configurations, but that is not required. For example, upper clamping member (910) and/or lower clamping member (920) may be generally straight or have any other suitable configuration.

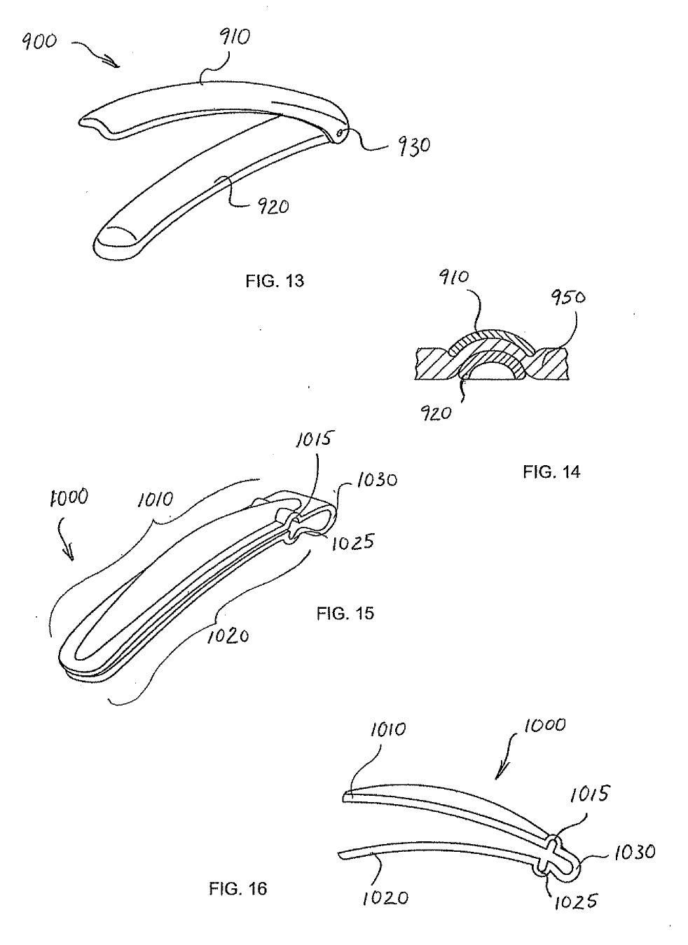

FIG. 14 depicts occlusion device (900) in a closed configuration with an anatomical structure (950) positioned between upper clamping member (910) and lower clamping member (920). As shown in FIGS. 13-14, upper clamping member (910) is configured to have a generally concave cross-section, while lower clamping member (920) is configured to have a corresponding generally convex cross-section. However, other suitable cross-sections will be apparent to one skilled in the art. Upper clamping member (910) and lower clamping member (920) may comprise metal, plastic, such as, for example, polyglycolic acid, or any other suitable material. Additionally, upper clamping member (910) and lower clamping member (920) may comprise the same material or they may comprise different materials. In the illustrated version, hinge (930) comprises a pin. However, hinge (930) may comprise a pin, a rod or any other suitable device or method. Hinge (930) may include a spring (not shown) configured to urge occlusion device (900) into a closed configuration. Other variations in structure, use, and method of operation of occlusion device (900) will be apparent to those of ordinary skill in the art.

Another exemplary occlusion device (1000) is shown in FIGS. 15-16. In this example, occlusion device (1000) has a unitary construction and comprises an upper clamping member (1010) connected to a lower clamping member (1020) with an urging member (1030). Upper clamping member (1010) and lower clamping member (1020) are generally curved in this example. It will be appreciated that such curvature may compliment curvature of an anatomical structure to which occlusion device (1000) will be secured, or may otherwise facilitate or optimize engagement of occlusion device (1000) with an anatomical structure. Alternatively, upper clamping member (1010) and lower clamping member (1020) may be generally straight or have any other suitable configuration. In this example, urging member (1030) is configured such that a constant spring force is applied to upper clamping member (1010) and lower clamping member (1020) urging occlusion device (1000) toward a closed configuration, as shown in FIG. 15. In the illustrated version, upper clamping member (1010) includes an upper notch (1015) and lower clamping member (1020) includes a lower notch (1025). Upper notch (1015) may be positioned along the width of upper clamping member (1010) such that upper notch (1015) is positioned opposite lower notch (1025) when occlusion device (1000) is in a closed configuration. Upper notch (1015) and lower notch (1025) may be configured to facilitate deployment or application to tissue with a suitable tool (not shown). Of course, upper notch (1015) and lower notch (1025) are not required, and other suitable structures, configurations or devices may be included to facilitate deployment or application. Other variations in structure, use, and method of operation of occlusion device (1000) will be apparent to those of ordinary skill in the art.

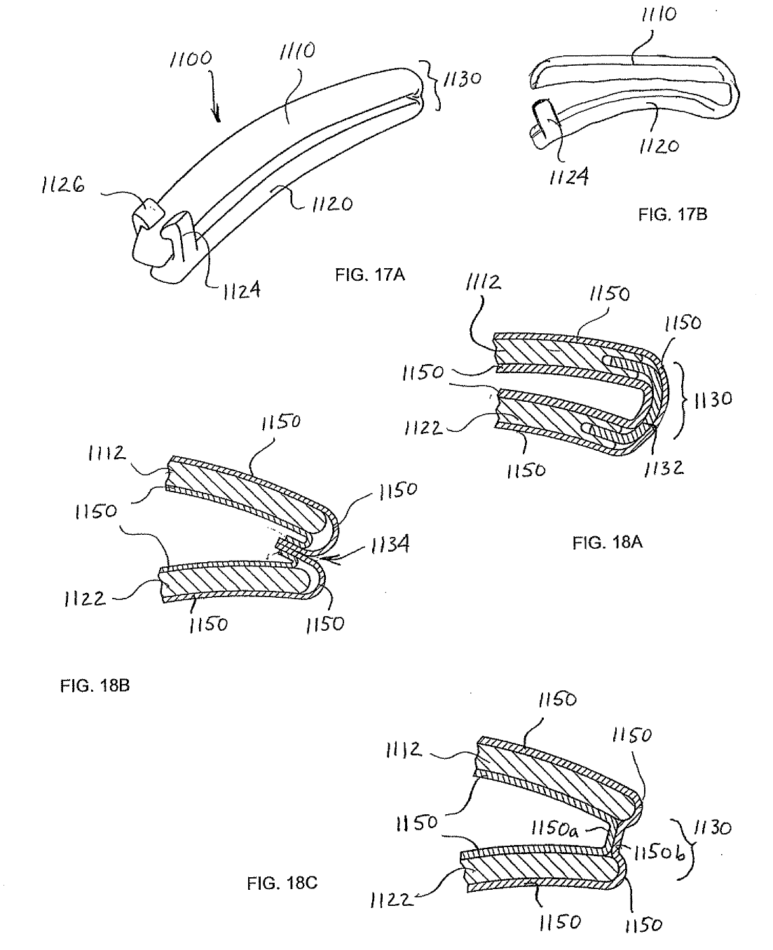

Further alternative embodiments of an occlusion device (1100) are shown in FIGS. 17A-18C. In this example, occlusion device (1100) comprises an upper clamping portion (1110) connected to a lower clamping portion (1120) with a hinge (1130). FIG. 17A depicts occlusion device (1100) in a closed configuration, while occlusion device (1100) is shown in an open configuration in FIG. 17B. All of the components of occlusion device (1100) may be enshrouded in a sock (1150), as described above. Sock (1150) may comprise a knit, braided polyester material. Of course, any other suitable materials may be used for sock (1150), including but not limited to polyethylene. Upper clamping portion (1110) may comprise a generally flat substantially rigid member (1112), while lower clamping portion (1120) may comprise a generally curved substantially flexible member (1122). In this example, lower clamping portion (1120) also includes a first clasping member (1124) and a second clasping member (1126) configured to releasably engage upper clamping portion (1110) when occlusion device (1100) is in a closed configuration.

In this version, hinge (1130) comprises a fabric hinge. As illustrated in FIGS. 18A-18C, hinge (1130) may comprise an inner hinge (1132), a fold back seam (1134), a double seam (1136) or any other suitable seam or device. In the version illustrated in FIG. 18A, inner hinge (1132) comprises a flexible member (1133) with a u-shaped configuration attached at one end to substantially rigid member (1112) and attached at the opposite end to substantially flexible member (1122). As shown in FIG. 18B, fold back seam (1134) comprises the end portions of sock (1150) folded substantially horizontally inward between upper clamping portion (1110) and lower clamping portion (1120), and secured together using stitching, adhesive, or any other suitable means. FIG. 18C depicts double seam (1136) which comprises an inner portion (1150a) of sock (1150) positioned adjacent to an outer portion (1150b) of sock (1150) in a substantially vertical orientation. Double seam (1136) may further comprise a stitching, or adhesive, or any other means for securing inner portion (1150a) to outer portion (1150b). Other variations in structure, use, and method of operation of occlusion device (1100) will be apparent to those of ordinary skill in the art.

Another exemplary occlusion device (1200) is shown in FIG. 19. In this example, occlusion device (1200) has a unitary construction and comprises an upper clamping member (1210) connected to a lower clamping member (1220) by a curved member (1230). Upper clamping member (1210) and lower clamping member (1220) are generally curved in this example. It will be appreciated that such curvature may compliment curvature of an anatomical structure to which occlusion device (1200) will be secured, or may otherwise facilitate or optimize engagement of occlusion device (1200) with an anatomical structure. Alternatively, upper clamping member (1210) and/or lower clamping member (1220) may be generally straight or have any other suitable configuration. Occlusion device (1200) may comprise plastic, such as, for example, polyglycolic acid, or any other suitable material. In the illustrated version, curved member (1230) follows a generally C-shape profile. However, curved member (1230) may have any suitable profile or configuration.

As shown in FIG. 19, upper clamping member (1210) includes a head (1212). In this example, head (1212) includes an aperture (1214) and a ratcheting member (1216). Lower clamping member (1220) may include a strap (1222), which may include serrations (1224). Serrations (1224) may occupy any suitable portion of strap (1222). In this example, ratcheting member (1216) is configured to engage serrations (1226) as strap (1224) is passed through aperture (1214). Occlusion device (1200) may be transitioned from an open configuration, as shown in FIG. 19, to a closed configuration (not shown) by passing strap (1224) through aperture (1214), thereby urging lower clamping portion (1220) toward upper clamping portion (1210). The pressure applied to an anatomical structure positioned between upper clamping member (1210) and lower clamping member (1220) may be adjusted by passing a longer portion of strap (1224) through aperture (1214). Once occlusion device (1200) is transitioned to a closed configuration, the portion of strap (1224) protruding beyond head (1212) may be severed from the portion of strap (1224) engaged by ratcheting member (1216). The transition of occlusion device (1200) from an open configuration to a closed configuration may result in a positive lock. It will be appreciated that head (1212) and strap (1222) may operate in a manner similar to a conventional “zip tie.” Other variations in structure, use, and method of operation of occlusion device (1200) will be apparent to those of ordinary skill in the art.

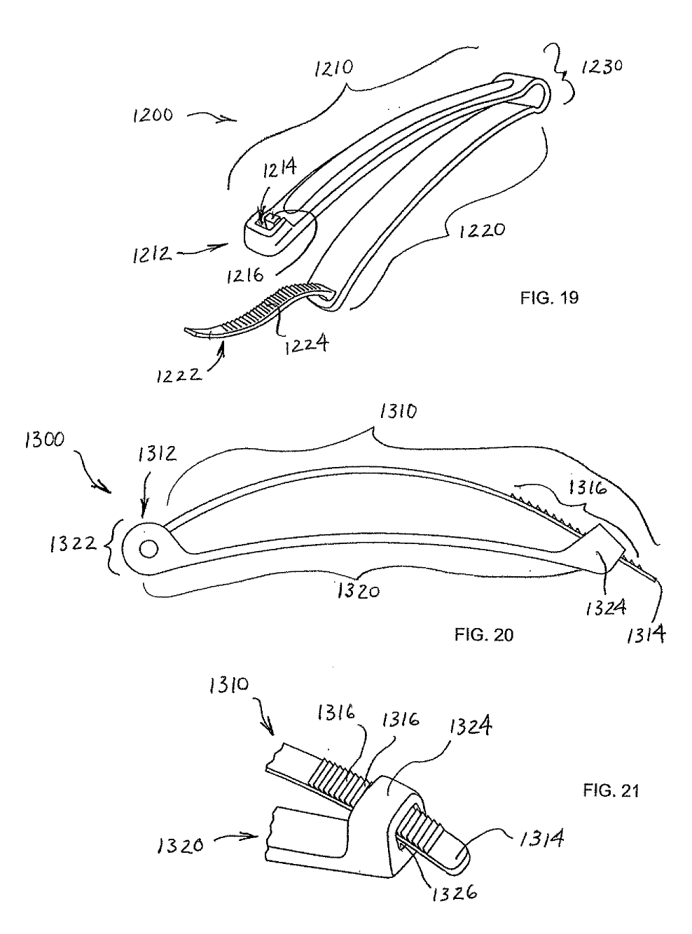

An alternative embodiment of an occlusion device (1300) is shown in FIGS. 20-21. In this example, occlusion device (1300) comprises a strap (1310) connected to a base member (1320). Base member (1320) may include a support portion (1322) and a head (1324). As shown in FIGS. 20-21, head (1324) includes an aperture (1326) and a ratcheting member (not shown). In this example, strap (1310) includes a fixed end (1312), a free end (1314), and serrations (1316). Fixed end (1312) may be attached to support portion (1322) using any suitable device or method. The ratcheting member (not shown) may be configured to engage serrations (1316) as free end (1314) is passed through aperture (1326). Head (1324) and strap (1310) may therefore operate in a manner similar to a conventional “zip tie.” The pressure applied to an anatomical structure positioned between strap (1310) and base member (1320) may be adjusted by passing a longer portion of strap (1310) through aperture (1326). Once occlusion device (1300) is transitioned to a closed configuration, the portion of strap (1310) protruding beyond head (1324) may be severed from the portion of strap (1310) engaged by the ratcheting member (not shown). The transition of occlusion device (1300) from an open configuration to a closed configuration may result in a positive lock. Other variations in structure, use, and method of operation of occlusion device (1300) will be apparent to those of ordinary skill in the art.

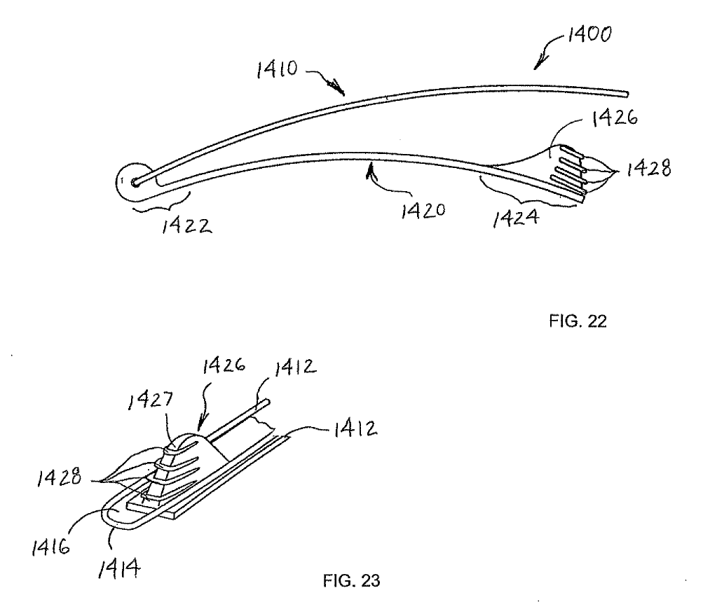

Another exemplary occlusion device (1400) is shown in FIGS. 22-23. In this example, occlusion device (1400) includes a latch (1410) hingedly connected to a base member (1420). Latch (1410) and base member (1420) are generally curved in this example. It will be appreciated that such curvature may compliment curvature of an anatomical structure to which occlusion device (1400) will be secured, or may otherwise facilitate or optimize engagement of occlusion device (1400) with an anatomical structure. Alternatively, latch (1410) and/or base member (1420) may be generally straight or have any other suitable configuration.

In the illustrated version, latch (1410) includes side members (1412), an end member (1414) and an opening (1416). As shown in FIGS. 22-23, latch (1410) has a generally u-shaped configuration. However, latch (1410) may have any suitable configuration. Base member (1420) may include a support portion (1422) and a latching portion (1424). As shown in FIG. 22, support portion (1422) is configured to hingedly attach latch (1410) and base member (1420). Latching portion (1424) may comprise a tab (1426) with ridges (1428) included on a front face (1427) of tab (1426). Ridges (1428) also extend along a portion of each side of tab (1426) in this example. As shown in FIGS. 22-23, tab (1426) has a generally curved configuration, however, tab (1426) may be generally curved, rectangular, triangular, or have any other suitable configuration. In this version, tab (1426) includes a set of four ridges (1428); however any suitable number of ridges may be used.

FIG. 22 depicts occlusion device (1400) in an open configuration, while occlusion device (1400) is shown in a closed configuration in FIG. 23. Occlusion device (1400) may be transitioned toward a closed configuration by partially or completely passing tab (1426) through opening (1416). In this example, ridges (1428) are configured to engage side members (1412) as tab (1426) is passed through opening (1416). Ridges (1428) may provide a range of locking positions configured to allow the user to vary the amount of pressure applied by occlusion device (1400). Other variations in structure, use, and method of operation of occlusion device (1400) will be apparent to those of ordinary skill in the art.

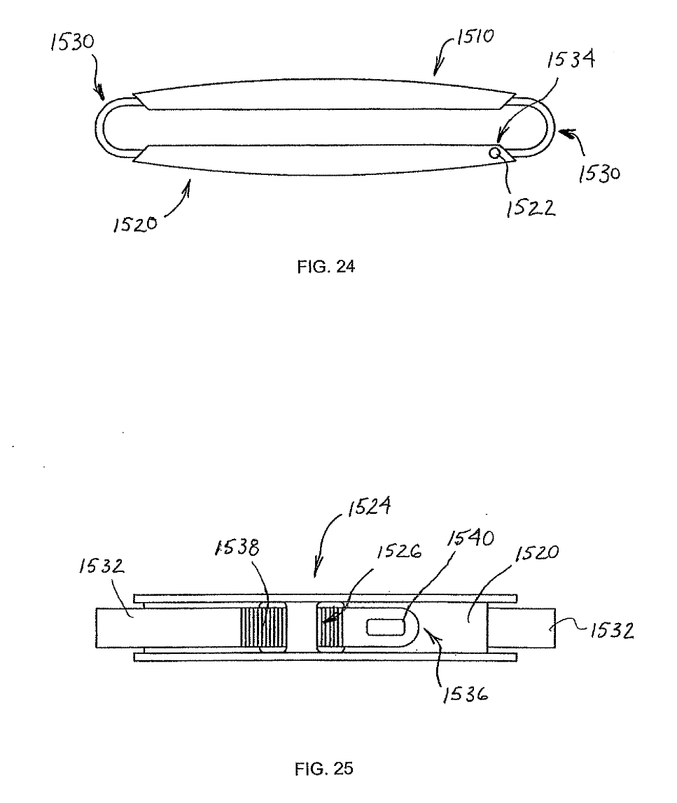

An alternative embodiment of an occlusion device (1500) is shown in FIGS. 24-25. In this version, occlusion device (1500) comprises an upper clamping member (1510), a lower clamping member (1520) and a locking mechanism (1530). As shown, lower clamping member (1520) includes a fastener (1522) and a locking member (1524) comprising an aperture (1526) and a ratcheting member (not shown). In this example, locking mechanism (1530) comprises a strap (1532) which includes a fixed end (1534), a free end (1536), serrations (1538) and an eyelet (1540). Strap (1532) may be sufficiently rigid to maintain a c-shaped profile between upper clamping member (1510) and lower clamping member (1520), as shown in FIG. 24. Serrations (1538) may occupy any suitable portion of strap (1532). Eyelet (1540) may be configured to facilitate grasping of strap (1532). As shown in FIGS. 24-25, fixed end (1534) is attached to lower clamping member (1520) by a fastener (1522) and looped around upper clamping member (1510) such that free end (1536) may be passed through locking member (1524). A ratcheting member (not shown) may be configured to engage serrations (1538) as strap (1532) is passed through aperture (1526). Locking member (1524) and strap (1532) may thus operate in a manner similar to a conventional “zip tie.” Occlusion device (1500) may be transitioned from an open configuration, as shown in FIG. 24, to a closed configuration (not shown) by grasping eyelet (1540) and passing strap (1532) through aperture (1526), thereby urging lower clamping member (1520) toward upper clamping member (1510). In an alternative version of this embodiment, the locking mechanism may comprise resilient urging members configured to connect the upper clamping member and lower clamping member while urging the upper clamping member and lower clamping member together. Other variations in structure, use, and method of operation of occlusion device (1500) will be apparent to those of ordinary skill in the art.

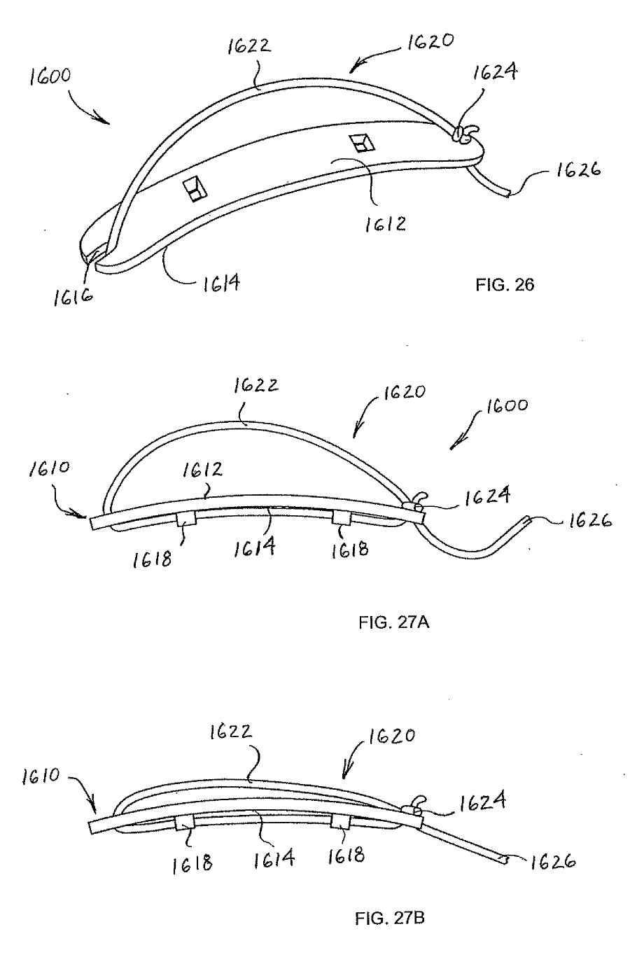

Another exemplary occlusion device (1600) is shown in FIGS. 26-27B. In this example, occlusion device (1600) comprises a support member (1610) and a flexible looped clamping member (1620). In the illustrated version, flexible clamping member (1620) comprises a suture. However, clamping member (1620) may comprise a suture, a thread, a strand, an absorbable fiber, a monofilament or any other suitable device. As shown in FIGS. 26-27B, flexible clamping member (1620) is looped around support member (1610), and flexible clamping member (1620) includes a looped portion (1622), a knot (1624), and an activation end (1626). Support member (1610) may include a clamping surface (1612), a lower surface (1614), a looping notch (1616), a fastening aperture (not shown), and alignment members (1618). In this version, looping notch (1616) and the fastening aperture (not shown) are located on opposite ends of support member (1610), and alignment members (1618) project outward from lower surface (1614). Alignment members (1618) each include an aperture (not shown). Support member (1610) is generally curved in this example. It will be appreciated that such curvature may compliment curvature of an anatomical structure to which occlusion device (1600) will be secured, or may otherwise facilitate or optimize engagement of occlusion device (1600) with an anatomical structure. Alternatively, support member (1610) may be generally straight or have any other suitable configuration.

FIGS. 26-27 A depict occlusion device (1600) in an open configuration, while FIG. 278 shows occlusion device (1600) in a closed configuration. Activation end (1626) of flexible clamping member (1620) may be passed through the fastening aperture (not shown), threaded along lower surface (1614) through the apertures (not shown) in alignment members (1618), passed through looping notch (1616), looped above clamping surface (1612), tied in a knot (1624) and passed through the fastening aperture (not shown). Occlusion device (1600) may be transitioned from an open configuration to a closed configuration by exerting a force on activation end (1626) in a direction generally away from support member (1610). In this example, knot (1624) is configured to allow activation end to be extended, thereby urging looped portion (1622) toward clamping surface (1612). The desired anatomical structure (not shown) may be occluded by positioning the anatomical structure between looped portion (1622) and clamping surface (1612) and transitioning occlusion device (1600) from an open configuration toward a closed configuration. To maintain a closed configuration, occlusion device (1600) may comprise a feature configured to permit movement of clamping member (1620) in only one direction. Alternatively, clamping member (1620) may be tied off, knotted, or otherwise manipulated to maintain a closed configuration. Other suitable techniques and features for maintaining a closed configuration will be apparent to those of ordinary skill in the art. Furthermore, other variations in structure, use, and method of operation of occlusion device (1600) will be apparent to those of ordinary skill in the art.

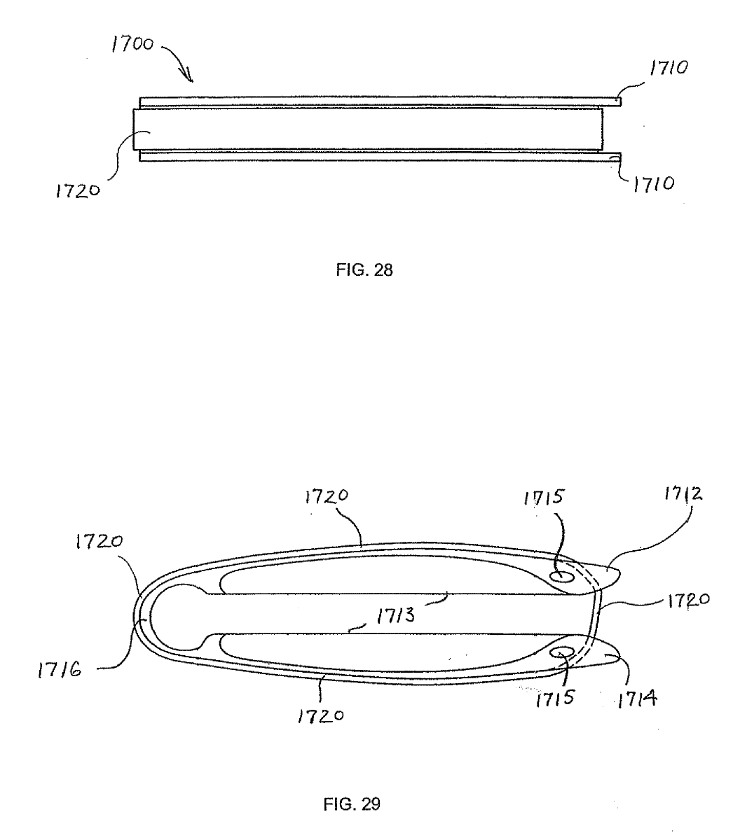

An alternative embodiment of an occlusion device (1700) is shown in FIGS. 28-29. In the illustrated version, occlusion device (1700) comprises a clamping structure (1710) and an urging device (1720). In this example, urging device (1720) is looped completely around the outer surface of clamping structure (1710), and urging device (1720) provides a constant inward force urging occlusion device (1700) toward a closed configuration (not shown). Urging device (1720) may comprise a rubber band, a resilient thread, an elastic loop, or any other suitable device. Clamping structure (1710) may comprise an upper clamping portion (1712), a lower clamping portion (1714) and a connecting portion (1716). As shown in FIGS. 28-29, upper clamping portion (1712) and lower clamping portion (1714) each include a substantially flat contact surface (1713) and application structures (1715) positioned adjacent the distal ends (1718) opposite connecting portion (1716). In this version, connecting portion (1716) has a generally c-shaped profile, however, any suitable configuration may be used. Application structures (1715) may be configured such that an applier (not shown) may engage application structures (1715) to facilitate manipulation of occlusion device (1700). Application structures (1715) may comprise apertures, indentations, recesses, protuberances, or any other suitable devices or structures. Of course, application structures (1715) may also be omitted. Other variations in structure, use, and method of operation of occlusion device (1700) will be apparent to those of ordinary skill in the art.

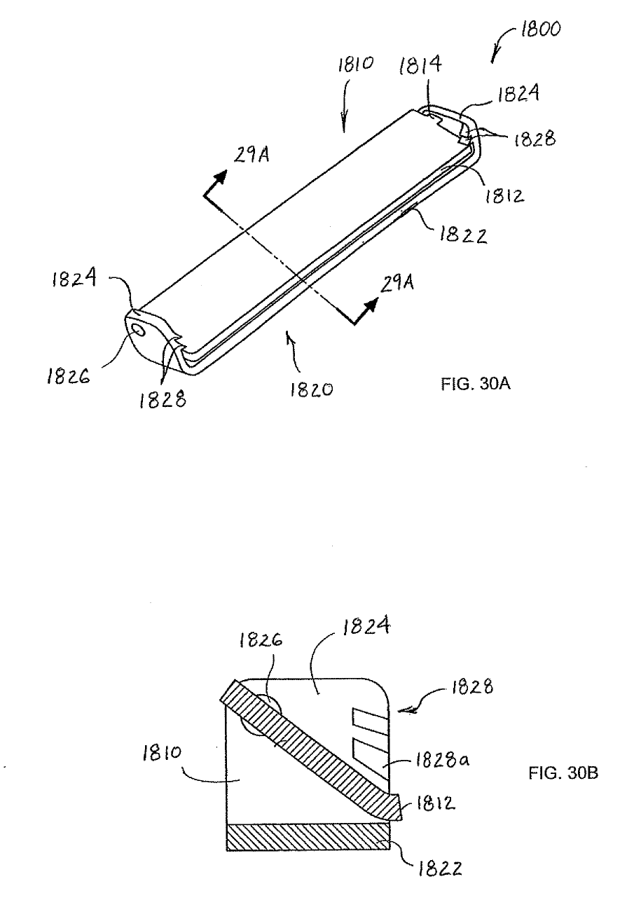

Another exemplary occlusion device (1800) is shown in FIGS. 30A-30B. In this example, occlusion device (1800) comprises a clamping plate (1810) and a base member (1820). Clamping plate (1810) may be generally flat with a slightly curved edge (1812) and may include attachment tabs (1814). Base member (1820) may comprise a substantially flat central portion (1822) and side portions (1824). In this example, side portions (1824) are oriented substantially perpendicularly to central portion (1822) and positioned on opposing edges of central portion (1822). Side portions (1824) may include attachment apertures (1826) and ratcheting teeth (1828). In the illustrated version, attachment tabs (1814) are inserted into attachment apertures (1826), such that clamping plate (1810) is rotatably connected to base member (1820). Clamping plate (1810) may rotate about a central axis extending generally through the center of attachment apertures (1826).

As shown in FIGS. 30A-30B, ratcheting teeth (1828) are configured to engage clamping plate (1810) as edge (1812) is rotated toward central portion (1822). Side portions (1824) may include a series of ratcheting teeth (1828) configured to provide multiple closed configurations, thereby enabling the user to vary the pressure applied to an anatomical structure positioned between clamping plate (1810) and central portion (1822). In the illustrated version, each side portion (1824) includes two ratcheting teeth (1828). However, any suitable number of ratcheting teeth (1828) may be used. The configuration and number of ratcheting teeth (1828) included on one side portion (1824) may correspond to the configuration and number of ratcheting teeth (1828) included on the opposing side portion (1824), but this is not required. Occlusion device (1800) may be regarded as being in an open configuration with clamping plate (1810) disengaged from ratcheting teeth (1828). With clamping plate (1810) engaged by a pair of intermediate ratcheting teeth (1828), occlusion device (1800) may be regarded as being in an intermediate clamping configuration. As shown in FIGS. 30A-30B, with clamping plate (1810) positioned between central portion (1822) and the adjacent pair of ratcheting teeth (1828a), occlusion device (1800) may be regarded as being in a closed configuration. Ratcheting teeth (1828) may be configured to substantially impede or completely prevent rotation of clamping plate (1810) toward an open configuration after ratcheting teeth (1828) engage clamping plate (1810). Other variations in structure, use, and method of operation of occlusion device (1800) will be apparent to those of ordinary skill in the art.

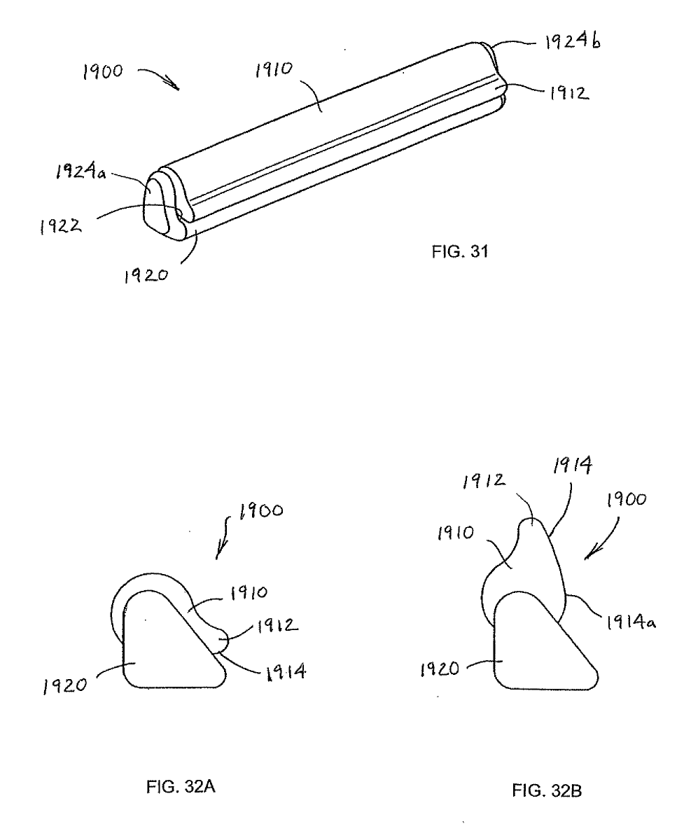

An alternative embodiment of an occlusion device (1900) is shown in FIGS. 31-32B. Occlusion device (1900) comprises a clamping member (1910) and a base member (1920). Clamping member (1910) may comprise a rotatable cam element, as shown in FIGS. 31-32B, including an elongated portion (1912) and a contact surface (1914). In particular, clamping member (1910) may be configured as an over-center cam. However, any suitable device or configuration may be used. In this example, base member (1920) includes a horizontal portion (1922) and side portions (1924a and 1924b). Base member (1920) may have a unitary configuration, but this is not required. In the illustrated version, clamping member (1910) is rotatably connected to side portions (1924a and 1924b) such that clamping member (1910) may be transitioned between a closed configuration, as shown in FIG. 32A, and an open configuration, as shown in FIG. 32B. Clamping member (1910) may be connected to side portions (1924a and 1924b) with a rod, tabs, or any other suitable device.

Occlusion device (1900) may be transitioned from an open configuration toward a closed configuration by rotating clamping member (1910) such that contact surface (1914) transitions toward horizontal portion (1922). Transitioning may be facilitated by an applier (not shown) configured to engage elongated portion (1912). Once occlusion device (1900) is transitioned toward a closed configuration with an appendage (not shown) positioned between clamping member (1910) and horizontal portion (1922), the appendage may apply a generally vertical horizontal force along the rear portion (1914a) of contact surface (1914), thereby securing occlusion device (1900) in a closed configuration. Other variations in structure, use, and method of operation of occlusion device (1900) will be apparent to those of ordinary skill in the art.

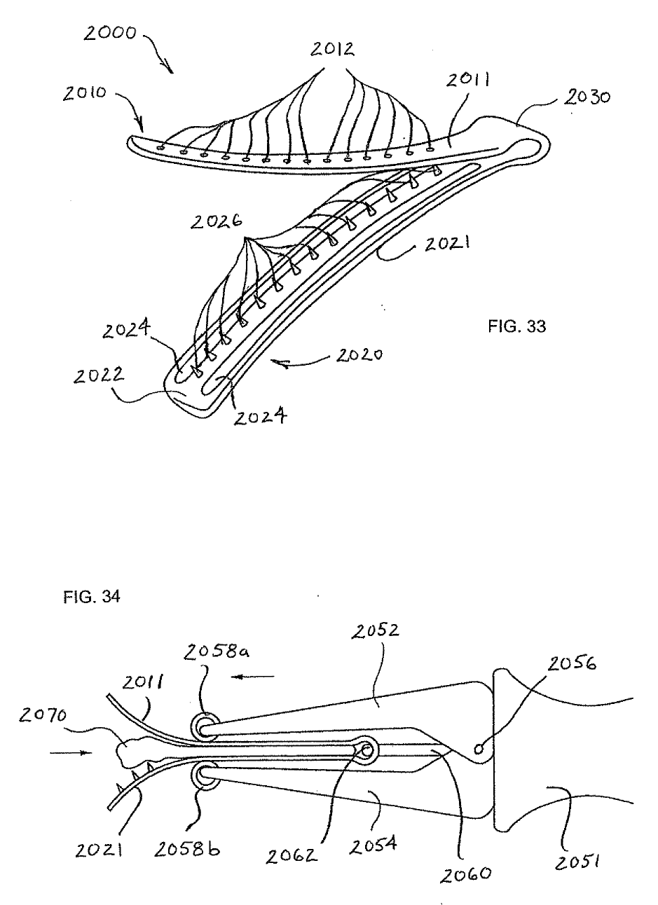

Another exemplary occlusion device (2000) is shown in FIGS. 33-34. In this example, occlusion device (2000) has a unitary construction and comprises an upper portion (2010), a barbed member (2020) and a connecting portion (2030). Occlusion device (2000) may comprise a substantially flexible material, such as, for example, polyglycolic acid, or any other suitable material. In this version, barbed member (2020) includes an outer surface (2021), an inner surface (2022), guide members (2024), and barbs (2026). In the illustrated version, barbed member (2020) includes fourteen barbs (2026) projecting outward from inner surface (2022) aligned along the longitudinal axis of barbed member (2020). However, barbed member (2020) may include any suitable number of barbs (2026) arranged in any suitable configuration. In this example, barbs (2026) comprise conical protuberances. However, barbs (2026) may comprise any suitable configuration, including, but not limited to, cylindrical posts and cylindrical posts with conical tips. Upper member (2010) includes an outer surface (2011) and attachment apertures (2012). As shown in FIG. 33, the number and configuration of attachment apertures (2012) corresponds to the number and configuration of barbs (2026). However, upper member (2010) may include any suitable number of attachment apertures (2012) in any suitable configuration. In this version, connecting portion (2030) attaches upper member (2010) to barbed member (2020), and connecting portion (2030) comprises a flexible hinge.

FIG. 34 depicts an applier (2050) facilitating the transition of occlusion device (2000) from an open configuration, as shown in FIG. 33, toward a closed configuration. Applier (2050) may include an upper jaw (2052), a lower jaw (2054), and a grasping member (2060) projecting from a main body (2051). In the illustrated version, upper jaw (2052) and lower jaw (2054) are connected by a hinge (2056), and each jaw includes a roller (2058a and 2058b). In this example, roller (2058a) is positioned adjacent to the distal end of upper jaw (2052) and roller (2058b) is positioned adjacent to the distal end of lower jaw (2054). Rollers (2058a and 2058b) are configured to travel along the outer surface (2011) of upper member (2010) and the outer surface (2021) of barbed member (2020). Grasping member (2060) includes a grasping tip (2062) configured to be inserted into connecting portion (2030) of occlusion device (2000).

As shown in FIG. 34, an anatomical structure (2070) may be positioned between upper member (2010) and barbed member (2020) when occlusion device (2000) is in an open configuration. Applier (2050) may be positioned such that grasping tip (2062) is inserted into connecting portion (2030) and rollers (2058a and 2058b) contact outer surface (2011) of upper member (2010) and outer surface (2021) of barbed member (2020) respectively. In order to transition occlusion device (2000) from an open configuration to a closed configuration, grasping member (2060) is drawn in a substantially horizontal direction, while upper jaw (2052) and lower jaw (2054) are urged in the opposite substantially horizontal direction. As occlusion device (2000) passes between rollers (2058a and 2058b) upper member (2010) and barbed member (2020) are drawn together, thereby urging barbs (2026) to protrude, at least partially, into attachment apertures (2012). Barbs (2026) may pierce anatomical structure (2070), but that is not required. Once a suitable length of occlusion device (2000) has passed through rollers (2058a and 2058b) occlusion device (2000) may be regarded as being in a closed configuration. Other variations in structure, use, and method of operation of occlusion device (2000) will be apparent to those of ordinary skill in the art.

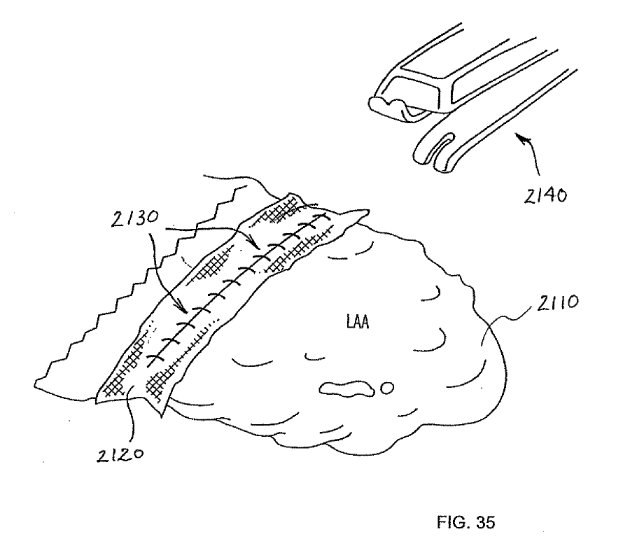

FIG. 35 depicts an alternative device and method of occluding an anatomical structure (2110). In the illustrated example, anatomical structure (2110) comprises the left atrial appendage. However, anatomical structure (2110) may comprise any suitable anatomical structure. The illustrated method includes a patch (2120), sutures (2130) and a deployment device (2140). Patch (2120) is configured to cover a portion of the upper surface (2111) of anatomical structure (2110). As shown in FIG. 35, patch (2120) covers the portion of anatomical structure (2010) that the user intends to suture. Patch (2120) may comprise a natural material, a synthetic material, such as, for example, polyester, or any other suitable material. In this example, a series of sutures (2130) is applied to the portion of anatomical structure (2110) covered by patch (2120), thereby, at least temporarily, securing patch (2120) to anatomical structure (2110). Sutures (2130) may be applied with a deployment device (2140). In this example, deployment device (2140) is an automated suturing device. However, sutures (2130) may be applied manually with a suturing needle or by any other suitable device or method. Sutures (2130) may be applied using any suitable technique, including, but not limited to, continuous stitch, interrupted stitch, mattress stitch, and blanket stitch. In addition, sutures (2130) may comprise a nonabsorbable synthetic material, such as, for example, nylon or polypropylene, an absorbable synthetic material, such as, for example, polyglycolic acid, a natural material, such as, for example, silk, or any other suitable material. Patch (2120) may provide structural support for sutures (2130) or serve any other purpose. Other variations in structure, use, and method of operation of patch (2120) and/or sutures (2130) will be apparent to those of ordinary skill in the art.

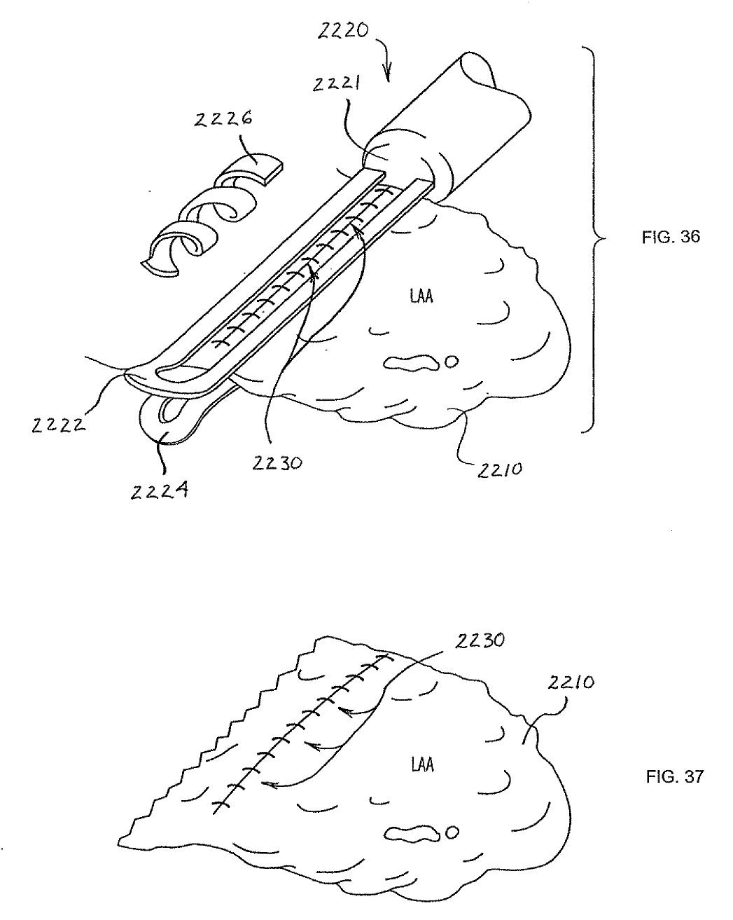

An alternative method and associated device for occluding an anatomical structure (2210) are depicted in FIGS. 36-37. Similar to the method and device illustrated in FIG. 34, as shown in FIGS. 36-37, anatomical structure (2210) comprises the left atrial appendage. However, anatomical structure (2210) may comprise any suitable anatomical structure. The illustrated method includes a deployment device (2220) and sutures (2230). Deployment device (2220) may include a front face (2221), an upper grasping portion (2222), a lower grasping portion (2224), and a spiral suturing member (2226). In this example, with anatomical structure (2210) positioned between upper grasping portion (2222) and lower grasping portion (2224), spiral suturing member (2226) extends from front face (2221) and penetrates into anatomical structure (2210). Spiral suturing member (2226) rotates as it passes through anatomical structure (2210), thereby applying sutures (2230) in a corkscrew or spiral configuration. As shown in FIGS. 36-37, sutures (2230) are applied along the entire width of anatomical structure (2210), but that is not required. Sutures (2230) may be applied to any suitable portion of anatomical structure (2210). Sutures (2230) may comprise an absorbable synthetic material, such as polyglycolic acid, or any other suitable material. The illustrated method and device may be particularly useful for anatomical structures with varying tissue thickness, and the illustrated method and device may help reduce or eliminate; trauma to adjacent tissue. Other variations in structure, use, and method of operation of deployment device (2220) will be apparent to those of ordinary skill in the art.

Another exemplary occlusion device (2300) is shown in FIGS. 38-39. In this version, occlusion device (2300) includes an upper clamping portion (2310), a lower clamping portion (2320), a hinge (2330), and a fluid delivery system (2340). As shown in FIGS. 38-39, upper clamping portion (2310), lower clamping portion (2320), and hinge (2330) are enshrouded in a sock (2350). Sock (2350) may comprise a knit, braided polyester material. Of course, any other suitable materials may be used for sock (2350), including but not limited to polyethylene. In this example, hinge (2330) comprises a fabric hinge formed by sock (2350). Upper clamping portion (2310) may comprise an insert (2312) enshrouded in sock (2350) Insert (2312) may comprise a polymer, such as, for example, polyglycolic acid, or any other suitable material. Insert (2312) may be substantially flat, substantially curved to correspond to the curvature of a particular anatomical structure, or have any other suitable configuration. Insert (2312) may be substantially rigid, but that is not required.

As shown in FIGS. 38-39, lower clamping portion (2320) comprises insert (2322), a bladder (2324), an inflation aperture (2325), a first clasping member (2326), and a second clasping member (2328), all of which are enshrouded in sock (2350). Insert (2322) may comprise a polymer, such as, for example, polyglycolic acid, or any other suitable material. Insert (2322) may be substantially flat, substantially curved to correspond to the curvature of a particular anatomical structure, or have any other suitable configuration. Insert (2322) may be substantially rigid, but that is not required. In the illustrated version, insert (2322) includes an upper surface (2323). Insert (2322) may be configured to allow bladder (2324) to rest adjacent to upper surface (2323). Bladder (2324) may be configured to expand and contract as fluid is delivered into and out of bladder (2324). In this example, first clasping member (2326) and second clasping member (2328) are configured to releasably engage upper clamping portion (2310) when occlusion device (2300) is in a closed configuration. Fluid may be delivered to bladder (2324) when occlusion device (2300) is in an open configuration or when occlusion device (2300) is in a closed configuration.

As shown in FIG. 38, fluid delivery system (2340) includes a delivery tube (2342) and a tube clip (2344). Fluid delivery system (2340) may include a luer fitting (not shown) configured to enable saline to be delivered to bladder (2324), but that is not required. In this example, delivery tube (2342) is passed through inflation aperture (2325) and connected to bladder (2324). Delivery tube (2342) may be configured to allow bladder (2324) to be inflated or deflated. With occlusion device (2300) in a closed configuration, the pressure applied to an appendage positioned between upper clamping portion (2310) and lower clamping portion (2320) may be varied by inflating or deflating bladder (2324). Once the desired level of inflation is reached, tube clip (2344) may be applied to delivery tube (2342) to temporarily seal delivery tube (2342) and maintain the desired level of inflation. In this version, tube clip (2344) comprises a u-shaped configuration, but any suitable configuration may be used. Tube clip (2344) may comprise a polymer, such as, for example, polyglycolic acid, or any other suitable material. Other variations in structure, use, and method of operation of occlusion device (2300) will be apparent to those of ordinary skill in the art.

An alternative embodiment of an occlusion device (2400) is shown in FIGS. 40-42. In this example, occlusion device (2400) comprises an upper clamping portion (2410) connected to a lower clamping portion (2420) with a hinge (2430), and a fluid delivery system (2450). Upper clamping portion (2410) and lower clamping portion (2420) are generally curved in this example. It will be appreciated that such curvature may compliment curvature of an anatomical structure to which occlusion device (2400) will be secured, or may otherwise facilitate or optimize engagement of occlusion device (2400) with an anatomical structure. Alternatively, upper clamping portion (2410) and lower clamping portion (2420) may be generally straight or have any other suitable configuration. Upper clamping portion (2410) includes an upper shell (2412) and an upper bladder (2414). In this version, upper shell (2412) includes a cavity configured to house upper bladder (2414), an attachment tab (2416) positioned on a distal end and a proximal end attached to hinge (2430). With upper bladder (2414) positioned in the cavity, a lower surface (2415) of upper bladder (2414) is exposed. Similarly, lower clamping portion (2420) includes a lower shell (2422) and a lower bladder (2424). In this version, lower shell (2422) includes a cavity configured to house lower bladder (2424), an attachment member (2426) positioned on a distal end and a proximal end attached to hinge (2430). With lower bladder (2424) positioned in the cavity, an upper surface (2425) of lower bladder (2424) is exposed. In this example, attachment member (2426) includes a slot (2427) configured to releasably engage attachment tab (2416) when occlusion device (2400) is in a closed configuration, as shown in FIGS. 41-42.

As shown in FIGS. 40-42, fluid delivery system (2450) comprises a delivery tube (2452) and a connection fixture (2454). Connection fixture (2454) may comprise a luer fitting or any suitable type of fitting. Fluid delivery system (2450) may deliver any suitable type of fluid, including, but not limited to, a gas, water, saline, or any other suitable fluid. In the illustrated example, delivery tube (2452) is connected to hinge (2430). Hinge (2430) may comprise a passageway through which fluid may pass from delivery tube (2452) through upper shell (2412) into upper bladder (2414) and through lower shell (2422) into lower bladder (2424). Alternatively, delivery tube (2452) may be connected directly to upper bladder (2414) and lower bladder (2424), or have any other suitable connection or configuration. In another embodiment (not shown), each bladder (2414 and 2424) is associated with a respective dedicated delivery tube.

The inflation level of upper bladder (2414) and/or lower bladder (2424) may be increased or decreased with occlusion device (2400) in an open configuration, as shown in FIG. 40, or closed configuration, as shown in FIGS. 41-42. As shown in FIG. 41, upper bladder (2414) and lower bladder (2424) are deflated or only partially inflated while occlusion device (2400) is in a closed configuration. FIG. 42 depicts occlusion device (2400) in a closed configuration with upper bladder (2414) and lower bladder (2424) both fully inflated, such that inner surface (2415) abuts inner surface (2425). The pressure applied to an appendage positioned between inner surface (2415) and inner surface (2425) may be varied by adjusting the inflation level of upper bladder (2414) and/or lower bladder (2424). Other variations in structure, use, and method of operation of occlusion device (2400) will be apparent to those of ordinary skill in the art.

FIG. 43 depicts an alternative version of an applier discussed in International Application Serial No. PCT/US2006/027553, published as WO 2007/009099, filed on Jul. 14, 2006, the disclosure of which has been incorporated by reference herein. As shown in FIG. 43, applier (2500) includes an alternative grasping portion (2510). As shown, grasping portion (2510) is similar to the grasping portion described in International Application Serial No. PCT/US2006/027553, except that lower rod (2520) of grasping portion (2510) has a telescoping portion (2522). Accordingly, by extending or retracting telescoping portion (2522), a user may change the effective length of lower rod (2522). For instance, such a grasping portion (2510) may be useful to accommodate occlusion devices of various lengths. By way of example only, an occlusion device may be approximately 25 mm long, or of any other length. It will also be appreciated that grasping portion (2510) need not necessarily be used to accommodate occlusion devices of varying lengths, and that the non-telescoping grasping portion described in International Application Serial No. PCT/US2006/027553 may also accommodate occlusion devices of varying lengths. In yet another embodiment (not depicted), upper rod (2530) also comprises a telescoping portion (not shown). Alternatively, upper rod (2530) may have a telescoping portion with lower rod (2520) lacking a telescoping portion. In still another embodiment, upper rod (2530) and/or lower rod (2520) may be configured such that their effective lengths may be varied using any suitable alternative to a telescoping portion. Other suitable variations will be apparent to those of ordinary skill in the art.

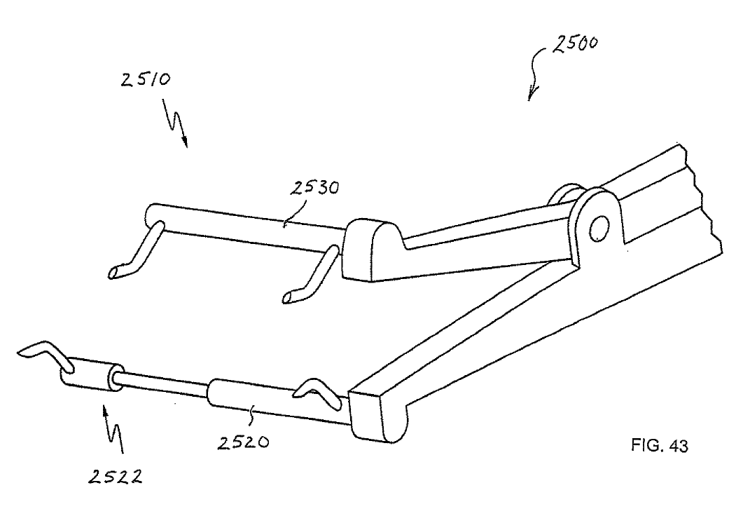

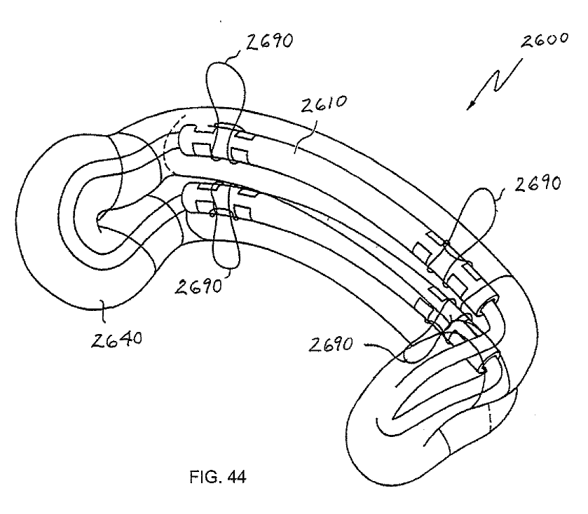

FIG. 44 depicts an alternative version of an occlusion device discussed in International Application Serial No. PCT/US2006/027553, published as WO 2007/009099, filed on Jul. 14, 2006, the disclosure of which has been incorporated by reference herein. It will be appreciated that an occlusion device (2600) may include one or more features configured to facilitate manipulation of the occlusion device (2600) by an applier. One example of such a feature is shown in FIG. 44. In particular, occlusion device (2600) is shown with a plurality of loops (2690). Loops (2690) are positioned on horizontal members (2610), and the spacing of loops (2690) is configured to correspond with the spacing of prongs on an applier. In this embodiment, for engagement of occlusion device (2600) with an applier, the prongs need not necessarily be inserted between horizontal members (2610); but can instead be inserted through corresponding loops (2690). With the prongs inserted in loops (2690), the handle portion of the applier may be squeezed by the user to bring the handle portion to a closed configuration, thereby bringing the grasping portion and occlusion device (2600) to an open configuration. In other words, the prongs may pull horizontal members (2610) apart by pulling on loops (2690). Loops (2690) may be provided as a part of a sock (2640) or may be provided separately (e.g., even in embodiments lacking a sock (2640)). Of course, as with any other component described herein, loops (2690) are merely optional, and loops (2690) may be subject to any other uses, modifications, substitutions, or supplements.

Still further embodiments are described in the appendix hereto.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A device for occluding an anatomical structure comprising:
at least first and second clamp portions adapted to cooperate to move from an open position that permits placement of the device adjacent an anatomical structure to a closed position that engages and occludes the anatomical structure.

2. The device of claim 1, wherein the at least first and second clamp portions comprise first and second beams.

3. The device of claim 1, wherein the at least first and second clamp portions comprise a beam and a flexible member.

4. The device of claim 1, wherein the at least first and second clamp portions are connected in a manner that forms a loop.

5. The device of claim 1, wherein the at least first and second portions are hingedly connected.

6. The device of claim 1, further comprising a fabric covering that covers at least one of the at least first and second clamp portions.

7. The device of claim 1, wherein at least one of the at least first and second clamp portions includes barbs adapted to pierce tissue.

8. A system for occluding an anatomical structure comprising:
a fluid expulsion portion,
a fluid transmitting portion,
a fluid adapted to be expelled from the fluid expulsion portion to provide occlusion of an anatomical structure.

9. The system of claim 8, wherein the fluid is a liquid substance.

10. The system of claim 8, wherein the fluid transmitting portion transmits the fluid to a position within the anatomical structure.

11. The system of claim 8, wherein the fluid transmitting portion transmits the fluid to a fluid retention portion that engages the exterior of the anatomical structure and the fluid is a gaseous substance.

12. An applicator for deploying an occlusion device, the applicator comprising:
a handle,
arms connected to the handle and being adapted to engage an occlusion device,
the arms having multiple portions that permit adjustment to engage occlusion devices having different sizes.

13. The system of claim 13, wherein the arms have telescoping portions.
